SEQUENCE LISTING (10 sequences)

SEQ ID NO: 1 - DNA, 47 bp, Artificial Sequence
  primer
ggggaattcatggatgctggaaatgctagaatataatcactatcagg

SEQ ID NO: 2 - DNA, 36 bp, Artificial Sequence
  primer
ggggcggccgcctaacaagtgtgctccgtctcttcc

SEQ ID NO: 3 - DNA, 57 bp, Artificial Sequence
  mitf19
ccggccaacttctttcatcaggaaactcgagtttcctgatgaaagaagttggttttt

SEQ ID NO: 4 - DNA, 57 bp, Artificial Sequence
  mitf20
ccggcggcatttgttgctcagaatactcgagtattctgagcaacaaatgccgttttt

SEQ ID NO: 5 - DNA, 57 bp, Artificial Sequence
  mitf22
ccggcgtggactatatccgaaagttctcgagaactttcggatatagtccacgttttt

SEQ ID NO: 6 - DNA, 57 bp, Artificial Sequence
  mitf23
ccggcgggaaacttgattgatctttctcgagaaagatcaatcaagtttcccgttttt

SEQ ID NO: 7 - DNA, 21 bp, Artificial Sequence
  primer
caggcatgaacacacattcac

SEQ ID NO: 8 - DNA, 20 bp, Artificial Sequence
  primer
tccatcaagcccaagatttc

SEQ ID NO: 9 - DNA, 18 bp, Artificial Sequence
  primer
ccaaccgcgagaagatga

SEQ ID NO: 10 - DNA, 19 bp, Artificial Sequence
  primer
ccagaggcgtacaggatag

(19) United States
(12) Patent Application Publication
(10) Pub. No.: US 2017/0027940 A1
(43) Pub. Date: Feb. 2, 2017

(54) METHOD FOR TREATING CANCER

(71) Applicant: STICHTING HET NEDERLANDS KANKER INSTITUUT, Amsterdam (NL)

(72) Inventors: Daniel Simon Peeper, Amsterdam (NL); Judith Nadja Mueller, Amsterdam (NL)

(21) Appl. No.: 15/302,787

(22) PCT Filed: Apr. 10, 2015
(86) PCT No.: PCT/NL2015/050237

§ 371 Date: Oct. 7, 2016

(30) Foreign Application Priority Data
Apr. 10, 2014 (NL) 2012605

(51) Int. Cl. A61K 31/506; C12Q 1/68; G01N 33/574; A61K 45/06

(52) U.S. Cl. CPC A61K 31/506; A61K 45/06; C12Q 1/6886; G01N 33/57496; C12Q 2600/106; C12Q 2600/158; G01N 2333/91205; G01N 2333/4706

(57) ABSTRACT

The current disclosure relates to pharmaceutical combinations and compositions useful in the treatment of certain types of cancer. The disclosure also relates to methods for treatment of these types of cancer. In particular, the disclosure relates to the combined use of of an inhibitor of a protein of the MAPK/ERK pathway and an inhibitor of specific kinases in the treatment of a cancer, in particular melanoma, in a patient. In an important embodiment, the cancer is characterized by the absence or reduced expression of MITF.

### FIELD OF THE INVENTION

The current disclosure relates to pharmaceutical combinations and compositions useful in the treatment of certain types of cancer. The disclosure also relates to methods for treatment of these types of cancer. In particular, the disclosure relates to the combined use of of an inhibitor of a protein of the MAPK/ERK pathway and an inhibitor of specific kinases in the treatment of a cancer, in particular melanoma, in a patient. In an important embodiment, the cancer is characterized by the absence or reduced expression of MITF.

### PRIOR ART

Cancer is one of the leading causes of death in the Europe and the United States. Despite recent advances in understanding mechanisms involved in cancer and in diagnosis and treatment, drug therapies for metastatic disease are often palliative in nature. Drug therapies seldom offer a long-term cure. There is a constant need for new methods of treatment, either in the form of monotherapy or in the form of combination treatment, combining different new or known drugs as first line therapy, and as second line therapies in treatment of resistant tumours.

Cancer cells are by definition heterogeneous. For example, multiple mutational mechanisms may lead to the development of cancer and mutational mechanisms associated with some cancers may differ between one tissue type and another; it is therefore often difficult to predict whether a specific cancer will respond to a specific chemotherapeutic (Cancer Medicine, 5th edition, Bast et al, B. C. Decker Inc., Hamilton, Ontario).

The treatment of cancer is gradually changing from an organ-centred to a pathway-centred approach. Cancer cells often have an addiction to the signals that are generated by the cancer-causing genes. Consequently, targeted cancer drugs that selectively inhibit the products of activated oncogenes can have dramatic effects on cancer cell viability. This approach has yielded significant clinical results for Non Small Cell Lung Cancer (NSCLC) having activating mutations in EGFR. However, this approach has not been successful in all type of cancers, for example in cancers characterized by oncogenic mutations in one of the

As an example, melanoma is a malignant tumor of melanocytes. It is one of the rarest forms of skin cancer but accounts for the majority of skin cancer deaths. Despite many years of intensive research, the only effective treatment is surgical resection of the primary tumor before it reaches a thickness of more than 1 mm. According to a WHO report, there are approximately 48,000 melanoma deaths each year, and about 160,000 new cases of melanoma are diagnosed yearly. It occurs more frequent in women than in men and is particularly common among Caucasians living in sunny climates, with high rates of incidence in Australia, New Zealand, North America, Latin America, and northern Europe.

Treatment of melanoma typically includes surgical removal of the melanoma, adjuvant treatment, chemo- and immunotherapy, and/or radiation therapy. The chance of a cure is greatest when the melanoma is discovered while it is still small and thin, and can be removed entirely.

Approximately 40-60% of (cutaneous) melanomas carry a mutation in the protein kinase referred to as BRAF. Approximately 90% of these mutations result in the substitution of glutamic acid for valine at codon 600 (BRAF V600E) although other mutations are also known (e.g. BRAF V600K and BRAF V600R). Such mutations in BRAF typically leads to proliferation and survival of melanoma cells (Davies et al Nature 2002; 417:949-54; Curtin et al N Engl J Med 2005;353:2135-47) through activation of the MAPK/ERK pathway. This pathway plays a significant role in modulating cellular responses to extracellular stimuli, particularly in response to growth factors, and the pathway controls cellular events including cell proliferation, cell-cycle arrest, terminal differentiation and apoptosis (Peyssonnaux et al., Biol Cell. 93(I-2):53-62 (2001)).

The discovery of the common BRAFV600E mutation in melanoma has resulted in the development of targeted therapies, which are associated with unprecedented clinical benefits. The small molecule inhibitor vemurafinib, specifically targeting the mutant BRAF kinase, for example, had become standard of care for patients diagnosed with mutant BRAF metastatic melanoma. Although this compound initially reduces tumour burden dramatically, eventually melanomas become resistant and patients progress in the disease (Wagle et al. J Clin Oncol. 29(22):3085-96 (2011)).

Resistance to the treatment appears the consequence of acquisition of additional mutations that affect the MAPK-signaling pathway. About 80% of the so far discovered resistance mechanisms to BRAF inhibition result in a phosphorylation of ERK and thereby reactivation of the oncogenic pathway. In addition, it was found that some BRAF-mutant melanoma tumors and cell lines that are resistant to RAF inhibition have harbour NRAS mutations (Wagle et al. J Olin Oncol. 29(22):3085-96 (2011)).

At the same time approximately 15% of BRAF mutant melanoma patients fail to respond to BRAF inhibition in the first place, owing to intrinsic resistance. BRAF-mutations are also found in other types of cancer.

Another example are NRAS mutations. Among the first oncogenes discovered in cutaneous melanoma was NRAS, which is mutant in up to 20% of tumours causing aberrant signalling in several downstream cascades. Despite, being a highly relevant therapeutic target, design of small molecules selectively inhibiting mutant NRAS in melanoma, to date, remains an unsolved challenge. The majority of NRAS mutations are found in codon 61 impairing the enzymatic activity of RAS to cleave GTP to GDP. Other, less frequent mutations are found in codon 12 and 13 preventing the association of GAPase activating proteins (GAP), which accelerate the weak hydrolytic potential of RAS. As a result, NRAS remains in its active, GTP-bound state driving cell proliferation, survival and motility making NRAS an important therapeutic target in melanoma (Posch, Oncotarget (2013) 4(4):494-5). NRAS mutation are also found in other types of cancer.

There is therefore a constant need for better understanding of the mechanisms that control and drive the development of cancer and for treatments directed thereto. It is therefore goal of the current invention to provide for new and improved methods of treatment of cancers, in particular KRAS, BRAF and NRAS-mutated cancers, as well as to provide for products and therapeutically pharmaceutical combinations for use in such (mutant) cancers. In addition it is a goal to provide for new and improved methods to better predict prognosis or response to treatment.

### DESCRIPTION OF THE DRAWINGS

FIG. 1: Exogenous MITF expression confers resistance to MAPK-pathway. BRAF mutant melanoma cells lines with high endogenous MITF levels were sensitized towards SCH772984 by shRNA-mediated knockdown of MITF. Exemplary immunoblot confirms knockdown of MITF leading to downregulation of MITF-targets CDK2 and MelanA and increased PARP-cleavage (indicated with an arrow) after ERK inhibition.

FIG. 2: MITF expression is frequently lost in resistant cells in vitro and in vivo. Immunoblot of sensitive (S) and resistant (R) melanoma cells shows expression of MITF and MITF-regulated proteins./pct

FIG. 3: MITF expression sharply dropped upon short-term exposure to PLX4720 and was further decreased in the remaining drug-tolerant population (DTP), drug-tolerant proliferating population (DTPP) and in resistant (R) cells.

FIG. 4: MitfmRNA expression was measured by q-RT-PCR in treatment-naïve and resistant melanoma cell lines normalized to Rp113 mRNA expression.

FIG. 5: Loss of MITF confers cross-resistance and increases invasiveness of resistant cells. Two PLX4270-resistant cells lines (Me1888 and SkMe128) and their treatment-naïve counterparts were treated with either ERKi, MEKi, the BRAFinhibitor Dabrafenib or a combination of the latter and stained with crystal violet after 6 days of treatment.

FIG. 6: Absence of MITF indicates innate insensitive cells in vitro and in vivo. Treatment-naïve BRAFV600E mutant melanoma cells were grouped based on their MITF expression in an immunoblot. The lower panel shows MITF-specific mRNA expression in different BRAFV600E mutant melanoma cells normalized to beta-actin.

FIG. 7: BRAFV600E mutant melanoma cells were plated in low density and treated with 5 μM PLX4270 for 6 days or left untreated and stained with crystal violet (left panel). For a subset of these cells a sufficient MAPK-pathway inhibition and cleaved PARP (indicated by arrow) was confirmed on immunoblot (right panel).

FIG. 8: MITF^{endo}^{_}^{high} and MITF^{endo}^{_}^{low} BRAF^{V600E} mutant melanoma cell lines were plated in low density and treated with either BRAFi, MEKi or a combination of those. After six days dishes were stained with crystal violet.

FIG. 9: An independent set of treatment-naïve BRAFV600E mutant melanoma cell lines was grouped on MITF expression and resistance towards BRAF inhibitor (vemurafenib) and ERK inhibitor determined by dose response curves. Cell lines with MITF amplification are marked with an asterix.

FIG. 10: Receptor Tyrosine Kinases are upregulated in MITF negative BRAF-mutant cells. : A phospho-RTK array was performed comparing one MITFendo_high and one MITFendo_low melanoma cell line untreated or treated with 5 μM PLX4270 for two days.

FIG. 11: AXL inhibition synergizes with BRAF inhibition in innate or acquired resistant cells. MITFendo_low cells were exposed to a combination of RTK inhibition (AXL, EGFR and/or PDGFRbeta) and MAPK-pathway inhibition. After nine days of combined treatment (as indicated) the remaining cells were stained with crystal violet.

FIG. 12: AXL-expressing PLX4270-resistant melanoma cells were exposed to 0.3 μM AXL inhibitor for nine days and the remaining cells stained with crystal violet.

FIG. 13: MITF low melanoma cell lines were exposed to inhibition BRAF (5 μM) with either AXIi (0.3 μM) (column 2) or EGFRi(2 μM) (column 3) or in a triple combination (Column 4; column 1 are control cells). After nine days of treatment the remaining cells were stained with crystal violet. The inhibitors were R428 for AXL, Gefitinib for EGFR and PLX4720 for BRAF. Results show that the triple combination surprisingly further inhibit proliferation/viability upon drug treatment, suggesting superior effect compared to the combination of a MAPK inhibitor and AXL inhibitor.

### DESCRIPTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. For example, conventional molecular biology, microbiology, pharmaceutical and recombinant DNA techniques are well known among those skilled in the art. Such techniques are explained fully in the literature.

For purposes of the present invention, the following terms are defined below.

As used herein, the singular forms “a,” “an” and “the” include plural referents unless the context clearly dictates otherwise. For example, a method for administrating a drug includes the administrating of a plurality of molecules (e.g. 10's, 100's, 1000's, 10's of thousands, 100's of thousands, millions, or more molecules).

As used herein, the term “and/or” indicates that one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . .

As used herein “cancer” and “cancerous” refers to or describes the physiological condition in mammals that is typically characterized by unregulated cell growth. The terms “cancer,” “neoplasm,” and “tumour,” are used interchangeably and refer to cells that have undergone a malignant transformation that makes them pathological to the host organism. Primary cancer cells can be distinguished from non-cancerous cells by techniques known to the skilled person. A cancer cell, as used herein, includes not only primary cancer cells, but also cancer cells derived from such primary cancer cell, including metastasized cancer cells, and cell lines derived from cancer cells. Examples include solid tumours and non-solid tumours or blood tumours. Examples of cancers include, without limitation, leukemia, lymphoma, sarcomas and carcinomas (e.g. colon cancer, pancreatic cancer, breast cancer, ovarian cancer, prostate cancer, lung cancer, melanoma, lymphoma, non-Hodgkin lymphoma, colorectal cancer, (malignant) melanoma, thyroid cancer, papillary thyroid carcinoma, lung cancer, non-small cell lung carcinoma, and adenocarcinoma of lung.

As is well known, tumours may metastasize from a first locus to one or more other body tissues or sites. Reference to treatment for a “neoplasm,” “tumour” or “cancer” in a patient includes treatment of the primary cancer, and, where appropriate, treatment of metastases.

As used herein, “in combination with” is intended to refer to all forms of administration that provide a first drug together with a further (second, third) drug. The drugs may be administered simultaneous, separate or sequential and in any order. Drugs administered in combination have biological activity in the subject to which the drugs are delivered. Within the context of the invention, a combination thus comprises at least two different drugs, and wherein one drug is at least an inhibitor of a protein of the MAPK/ERK pathway and wherein the other drug is at least an inhibitor of a kinase selected from the group consisting of AXL, EGFR, PDGFR, IGF-IR, EphA7, PDGFRbeta, EphA2 and Mer, as disclosed herein in detail. In an embodiment, in the combination, the inhibitor of a protein of the MAPK/ERK pathway is a selective inhibitor, and does preferably does not inhibit the kinase selected from the group consisting of AXL, EGFR, PDGFR, IGF-IR, EphA7, PDGFRbeta, EphA2 and Mer, as disclosed herein in detail. In an embodiment, in the combination, the inhibitor of the kinase selected from the group consisting of AXL, EGFR, PDGFR, IGF-IR, EphA7, PDGFRbeta, EphA2 and Mer is a selective inhibitor, and within the context of the current invention, does not inhibit a protein of the MAPK/ERK pathway. In a further embodiment, in the combination, both the inhibitor of a protein of the MAPK/ERK pathway and the inhibitor of the kinase selected from the group consisting of AXL, EGFR, PDGFR, IGF-IR, EphA7, PDGFRbeta, EphA2 and Mer, as disclosed herein in detail, are selective inhibitors.

A used herein “compositions”, “products” or “combinations” useful in the methods of the present disclosure include those suitable for various routes of administration, including, but not limited to, intravenous, subcutaneous, intradermal, subdermal, intranodal, intratumoral, intramuscular, intraperitoneal, oral, nasal, topical (including buccal and sublingual), rectal, vaginal, aerosol and/or parenteral or mucosal application. The compositions, formulations, and products according to the disclosure invention normally comprise the drugs (alone or in combination) and one or more suitable pharmaceutically acceptable excipients or carriers.

As used herein, “to comprise” and its conjugations is used in its non-limiting sense to mean that items following the word are included, but items not specifically mentioned are not excluded. It also encompasses the more limiting “to consist or.”

Within the context of the current disclosure, the combination of the at least one inhibitor of a protein of the MAPK/ERK pathway and the at least one inhibitor of a kinase selected from the group consisting of AXL, EGFR, PDGFR, IGF-IR, EphA7, PDGFRbeta, EphA2 and Mer are administrated in an effective amount. As used herein, “an effective amount” is meant the amount of the combination required to ameliorate the symptoms of a disease relative to an untreated patient. The effective amount of active agent(s) used to practice the present disclosure for therapeutic treatment of a cancer varies depending upon the manner of administration, the age, body weight, and general health of the subject. Ultimately, the attending physician will decide the appropriate amounts and dosage regimen. Such amount is referred to as an “effective” amount. Thus, in connection with the administration of a drug combination which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

As used herein, “expression level” or “amount of a protein” refers to the amount of a molecule expressed in a cell, and in connection therewith, with the amount of activity of the protein in the cell. The expression level of a protein can be represented by the amount of messenger RNA (mRNA) encoded by a gene, the amount of polypeptide corresponding to a given amino acid sequence encoded by a gene, the amount of biochemical forms of the proteins expressed in a cell, including the amount of particular post-synthetic modifications, including phosphorylation. For example, an expression level of a protein can be the amount of a particular form of the molecule such as the phosphorylated form of a polypeptide. Depending on the phosphorylation status, a protein may be activated or inactivated for a particular activity. Multiple forms of the protein may thus exist, for example, based on the phosphorylation state at different sites on the same polypeptide. The amount of each of these different biochemical forms is intended to be included in the meaning of an expression level or amount of a certain protein. The expression level can refer to an absolute amount of the molecule in a specimen or to a relative amount of the molecule. The expression level of a molecule can be determined relative to a control molecule in the specimen. The amount of a protein or the level of expression may be determined using assays and methods available in the prior art and well-known to the skilled person and include methods using DNA microarray hybridization, using polymerase chain reaction (PCR), determining levels of RNA, using a direct quantitation method such as the isotope-coded affinity tag (ICAT) methods, using immunostaining, techniques comprising enzyme-linked immunosorbent assay (ELISA), radioimmunoassay (RIA), sandwich assay, Western blotting, immunoblotting analysis, an immunohistochemistry method, use of phospho-specific antibodies, measuring enzymatic activity of the protein, or a combination thereof. These and other techniques are all well-known to the skilled person.

As used herein, in general the term “inhibitor” of a (defined) protein or enzyme, for example ERK, refers to any compound capable of down-regulating, decreasing, suppressing or otherwise regulating the amount and/or activity of the (defined) protein, for example ERK, for example, to a level of 50%, 30%, 20% or 10% or less compared to the control (without the presence of such inhibitor). Inhibitors may include, but are not limited to small molecules (chemical compound having a molecular weight below 2,500 daltons, more preferably between 300 and 1,500 daltons, and still more preferably between 400 and 1000 daltons), antibodies directed to the particular protein or enzyme, compounds that down-regulate gene expression, translation and/or transcription, including such RNA molecules capable of RNA interference including, without limitation, siRNA, shRNA, and miRNA. The inhibitors to be used in accordance with the present invention may be selective inhibitors of said (defined) protein; the term “selective” or “selectivity” expresses the biologic fact that at a given compound concentration enzymes (or proteins) are affected to different degrees. In the case of proteins selective inhibition can be defined as preferred inhibition by a compound at a given concentration. In other words, an enzyme is selectively inhibited over another enzyme when there is a concentration which results in inhibition of the first enzyme whereas the second enzyme is not affected. To compare compound effects on different enzymes it is important to employ similar assay formats. For the proteins/enzymes as disclosed herein, such assay formats are readily available in the prior art. Thus, within the context of the current invention the different drugs used in the combination may be drugs that selectively inhibit one of the proteins to be inhibited according to the invention in comparison to the other protein(s), for example when used in a clinical setting.

“Patient”, as used herein, refers to human subjects, but also includes non-human primates, and laboratory animals including rodents such as mice, rats and guinea pigs, and the like. The term does not denote a particular age or sex. Thus, adult and newborn subjects, whether male or female, are intended to be included within the scope of this term. Preferably the patient is human.

“Pharmaceutically acceptable” is employed herein to refer to those combinations of the therapeutic combinations as described herein, and other drugs or therapeutics, materials, compositions, and/or dosage forms which are, within the scope of sound medical judgment, suitable for use in contact with the tissues of human beings and animals, without excessive toxicity, irritation, allergic response, or other problem or complication, commensurate with a reasonable benefit/risk ratio.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”.

As used herein “simultaneous” administration refers to administration of more than one drug at the same time, but not necessarily via the same route of administration or in the form of one combined formulation. For example, one drug may be provided orally whereas the other drug may be provided intravenously during a patients visit to a hospital. “Separate” administration includes the administration of the drugs in separate form and/or at separate moments in time, but again, not necessarily via the same route of administration. “Sequentially” of “sequential administration” indicates that the administration of a first drug if followed, immediately or in time, by the administration of the second drug, but again, not necessarily via the same route of administration.

As used herein, the terms “treat,” treating”, “treatment,” and the like refer to reducing or ameliorating a disorder and/or symptoms associated therewith. It will be appreciated that treating a disorder or condition does not require that the disorder, condition or symptoms associated therewith be completely eliminated.

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Cancers that are either wild type or mutant for NRAS, KRAS or BRAF are identified by known methods. For example, wild type or mutant NRAS/BRAF/KRAS cancer cells can be identified by DNA amplification and sequencing techniques, DNA and RNA detection techniques, including, but not limited to Northern and Southern blot, respectively, and/or various biochip and array technologies. Wild type and mutant polypeptides can be detected by a variety of techniques including, but not limited to immunodiagnostic techniques such as ELISA, or Western blot.

Throughout this application, various references are cited in parentheses to describe more fully the state of the art to which this invention pertains. Full bibliographic information for each citation is found at the end of the specification, immediately preceding the claims. The disclosure of these references are hereby incorporated by reference into the present disclosure in their entirety.

### DETAILED DESCRIPTION

The current disclosure is based on the surprising finding that a combination of at least one inhibitor of a protein (enzyme) of the MAPK/ERK pathway and at least one inhibitor of a kinase selected from the group consisting of AXL, EGFR, PDGFR, IGF-IR, EphA7, PDGFRbeta, EphA2 and Mer, may be co-operative and/or synergistic, i.e. produces an effect greater than the effect of the individual drugs, or even greater than the sum of the their individual effects, in inhibiting proliferation, or inducing apoptosis, or in treating in a cancer, preferably melanoma, in a patient, preferably a human. It was found that such combination is in particular effective in cancers that are characterized by a reduced amount of MITF, i.e. that have a reduced expression of MITF, including those cancers in which no MITF can be detected using standard techniques including immuno-staining, and as exemplified in the Examples herein.

In addition, the combination is effective in cancers that, next to the absence of MITF, or a reduced amount of MITF, show the presence of, or increased amounts of selected kinases, i.e. kinases selected from the group consisting of AXL, EGFR, PDGFR, IGF-IR, EphA7, PDGFRbeta, EphA2 and Mer. In particular it was found that cancers with a reduced amount of MITF and an increased amount of AXL (as compared to cells defined as having reduced MITF expression, or absence of MITF expression) may advantageously be treated with the combinations of the current disclosure. In particular it is beneficial to treat a patent with at least one inhibitor of a protein of the MAPK/ERK pathway, with at least one inhibitor of AXL, and with inhibitors directed to one or more of the above receptor tyrosine kinases, in particular when, in addition to AXL, one or more of the other receptor tyrosine kinases in such patient is overexpressed (as compared to cells defined as having reduced MITF expression, or absence of MITF expression).

In one embodiment the cancer is selected from the group consisting of NRAS-, KRAS- and BRAF-mutated cancer, preferably NRAS-mutated cancer, for example, but not limited to NRAS-, KRAS- and BRAF-mutated melanoma, for example NRAS-mutated melanoma or lung cancer. The inhibitors in the combination may, in one embodiment be selective inhibitors, or a selective inhibitor. In addition, the claimed combination works particularly well in those cells that are relatively insensitive to inhibition by inhibitors of a protein of the MAPK/ERK pathway alone (e.g. a RAF-inhibitor alone, an ERK-inhibitor alone, a MEK-inhibitor alone). Such cells are also referred to as resistant cancer cells and do not normally respond to treatment. The cancer may be resistant at the beginning of treatment (often called intrinsic resistance), or it may become resistant during treatment (often called acquired resistance ,also called refractory cancer). In other words, in one embodiment the cancer is a NRAS-, KRAS- and BRAF-mutated cancer, preferably melanoma, that is or has become (relatively) insensitive or resistant to an inhibitor of a protein of the MAPK/ERK-pathway, preferably, that has become relatively insensitive or resistant to a ERK-inhibitor, a MEK-inhibitor, a and/or a RAF-inhibitor, i.e. has or acquired resistance. The term “acquired resistance” indicates that the cancer becomes resistant to the effects of the drug after being exposed to it for a certain period of time. In a further embodiment the cells of the cancer have or required resistance to inhibitors of AXL. In a further preferred embodiment the cells of the cancer, preferably melanoma, have or required resistance to inhibitors of AXL and to an inhibitor of a protein of the MAPK/ERK-pathway preferably, that has become relatively insensitive or resistant to a ERK-inhibitor, a MEK-inhibitor, a and/or a RAF-inhibitor.

The inventors of the present invention have demonstrated, via experiments, that a combination of an inhibitor of a protein of the MAPK/ERK pathway and an inhibitor of a kinase selected from the group consisting of AXL, EGFR, PDGFR, IGF-IR, EphA7, PDGFRbeta,

EphA2 and Mer manifests an unexpected and strong co-operative and/or synergistic, therapeutic effect on the treatment of cancers, in particular cancers with a reduced amount of MITF or a reduced amount of MITF and an increased amount of at least one of the kinases, in particular NRAS-, KRAS- and BRAF-mutated cancers, including melanoma.

The invention thus provides for improved treatment strategies by employing the combination at least two different drugs or compounds, directed to inhibiting the combination of proteins/enzymes as disclosed herein. This for the first time allows to optimize the drug treatment by specifically optimizing treatment so as to inhibit the combination of proteins/enzymes in the best possible way, for example by applying selective inhibitors. For example, by the combination, the dose of each of the drugs in the combination may be optimized in order to achieve optimal treatment effect. For example the individual dose of a first individual drug in the combination may be optimized to achieve optimal inhibition of a first protein, and a second, third or further drug in the combination may be optimized to achieve optimal inhibition of the other protein/enzyme to be inhibited, and as detailed herein. In addition, the invention allows for the treatment with various and different combinations of inhibitors of the proteins/enzymes to be inhibited, as detailed herein. This is very useful in case, for example, for an individual patient, certain drugs or drug combinations are not well tolerated or lead to undesired further complications. The current invention allows for the replacement of a drug in such combination, or of the combination by another drug combination, in accordance with the invention and in order to overcome undesired effects or, again optimize treatment of the patient. In addition, when using the combination, the dose of the individual drugs may be lowered compared to when the drugs are used individually, which may be beneficial in view of toxicity.

The combination disclosed herein exhibits (therapeutic) co-operation and/or synergy when used to treat a subject or patient. Such effect may be demonstrated by the showing that the combination is superior to one or other of the constituents used as at a given, for example, optimum dose.

In addition, the inventor found that an reduced amount of MITF, or absence of MITF, in a cell obtained from a cancer from a patient is predictive for the response to particular cancer treatments, and may therefore be used as a negative predictor for treatment outcome.

In a first aspect of the current disclosure, there is provided for a combination of an inhibitor of a protein of the MAPK/ERK pathway and an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL for use as a medicament, preferably for use in the treatment of a cancer, preferably melanoma, in a patient.

The inhibitor of a protein of the MAPK/ERK pathway may be any inhibitor that reduces the amount or activity of one or more proteins that belong to the MAPK/ERK pathway. The MAPK/ERK pathway is well-known to the skilled person and is one of the four parallel mitogen activated protein kinase (MAPK) signaling pathways identified: ERK1/ERK2, JNK, p38 and ERK5.

The pathways are involved in cellular events such as growth, differentiation and stress responses (J. Biol. Chem. (1993) 268, 14553-14556). These four pathways are linear kinase cascades in that MAPKKK phosphorylates and activates MAPKK, and MAPKK phosphorylates and activates MAPK. To date, seven MAPKK homologs (MEK1, MEK2, MKK3, MKK4/SEK, MEK5, MKK6, and MKK7) and four MAPK families (ERK1/2, JNK, p38, and ERK5) have been identified. Activation of these pathways regulates the activity of a number of substrates through phosphorylation. These substrates include: transcription factors such as TCF, c-myc, ATF2 and the AP-1 components, fos and Jun; cell surface components EGF-R; cytosolic components including PHAS-I, p90rsk, cPLA2 and c-Raf-1; and cytoskeleton components such as tau and MAP2. MAPK signaling cascades are involved in controlling cellular processes including proliferation, differentiation, apoptosis, and stress responses.

Of the known MAPK signaling pathways, the MAPK/ERK pathway (also referred to as RAF-MEK-ERK pathway or Ras-Raf-MEK-ERK pathway) mediates proliferative and anti-apoptotic signaling from growth factors and oncogenic factors such as Ras and Raf mutant phenotypes that promote tumor growth, progression, and metastasis. By virtue of its central role in mediating the transmission of growth-promoting signals from multiple growth factor receptors, the MAPK/ERK pathway provides molecular targets with potentially broad therapeutic applications in, for example, cancerous and none-cancerous hyperproliferative disorders, immunomodulation and inflammation.

Within the context of the current invention a protein of the MAPK/ERK pathway includes ERK, MEK, and RAF proteins, as discussed below.

In a preferred embodiment, the protein of the MAPK/ERK pathway is selected from the group consisting of RAF, MEK, and ERK, and combination of two, or three thereof. Thus in a preferred embodiment, the inhibitor of a protein of the MAPK/ERK pathway is selected from the group consisting of a RAF-inhibitor, an ERK-inhibitor, and a MEK-inhibitor, or combinations thereof.

In a preferred embodiment more than one inhibitor of a protein of the MAPK/ERK pathway is used. For example, two, three, or four inhibitors of one or more proteins of the

MAPK/ERK pathway are used in the combination therapy disclosed herein, i.e. in combination with an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL, or two, three or more of such kinase inhibitors. For example, at least one AXL-inhibitor may be combined with at least one MEK-inhibitor and/or at least one ERK-inhibitor, and/or at least one RAF-inhibitor. Or a combination of an AXL-inhibitor, a PDGFR inhibitor, an EGFT inhibitor can be combined in the treatment with, for example, an ERK-inhibitor, and/or a BRAF-inhibitor and/or a MEK-inhibitor.

A RAF protein, polypeptide or peptide is to indicate a polypeptide having serine/threonine protein kinase activity. RAF kinases are a family of three serine/threonine-specific protein kinases that are related to retroviral oncogenes. The three RAF kinase family members are ARAF (A-RAF; for example Genbank Accession NO: NP001243125), BRAF (B-RAF) and CRAF (C-RAF; (e.g. Gene accession number 5894; Refseq RNA Accessions NM_002880.3 ; protein NP_002871.1).

For example, BRAF (for example, Genbank Accession NO: NP004324) phosphorylates and activates MEK (MEK1 and MEK2). BRAF is a member of the RAF family, which includes ARAF and CRAF in humans (Ikawa, Mol Cell Biol. 8(6):2651-4 (1988)). BRAF is a serine/threonine protein kinase and participates in the RAS/RAF/MEK/ERK mitogen activated protein kinase pathway (MAPK pathway, see Williams & Roberts, Cancer Metastasis Rev. 13(1):105-16 (1994); Fecher et al 2008 Curr Opin Oncol 20, 183-189). CRAF acts as a MAP3 kinase, initiating the entire kinase cascade of the MAPK/ERK pathway.

These amino acid sequence of BRAF, CRAF and ARAF enzymes, other proteins mentioned herein, and variations thereof are available in GenBAnk, accessible via http://www.ncbi.nlm.nih.ciov/cienbank/ by entering either the numbers mentioned above or entering the relevant protein name.

By RAF (biological) activity is meant any function of RAF, such as enzymatic activity, kinase activity, or signaling the MAPK/ERK pathway.

By RAF inhibitor, for example a BRAF inhibitor, is meant a compound that reduces the biological activity of RAF, for example BRAF; or that reduces the expression of an mRNA encoding a RAF polypeptide, for example BRAF; or that reduces the expression of a RAF polypeptide, for example BRAF. RAF kinase inhibitors as used herein include efficient inhibitors of RAF kinase, particularly CRAF kinase inhibitors and wild and mutated BRAF kinase inhibitors, e.g. including inhibitors of mutant BRAF kinase.

RAF kinase inhibitors are known to the skilled person. Any RAF inhibitor, including any pharmaceutical agent having RAF inhibitory activity or selective RAF inhibitors may be utilized in the present invention.

Examples of RAF kinase inhibitors, including BRAF kinase inhibitors include the compounds GW5074, BAY 43-9006, CHIR-265 (Novartis), Vemurafenib, PLX4720 (Tsai et al. 2008 PNAS 105(8):3041) , PLX4032 (RG7204), GDC-0879 (Klaus P. Hoeflich et al. Cancer Res.2009 April 1;69:3042-3051), sorafenib tosylate (e.g. from Bayer and Onyx Pharmaceuticals as Nexavar), dasatinib (also known as BMS-354825, e.g. as produced by Bristol-Myers Squibb and sold under the trade name Sprycel), erlotinib (e.g. as marketed by Genentech and OSI pharmaceuticals as Tarceva), LGX818 from Novartis, dabrafenib (TafinlarTm capsule, made by GlaxoSmithKline, LLC), dabrafenib, gefitinib, imatinib mesilate, lapatinib, sunitinib malate, GSK2118436, benzenesulfonamide, N-[3-[5-(2-amino-4-pyrimidinyl)-2-(1,1-dimethylethyl)-4-thiazolyl]- 2-fluorophenyl]-2,6-difluoro-, methanesulfonate (1:1), N-{3-[5-(2-aminopyrimidin-4-yl)-2-(1,1-dimethylethyl)thiazol-4-yl]-2-fluorophenyl}-2,6-difluorobenzenesulfonamide monomethanesulfonate (Clin Cancer Res. 2011; doi: 10.1158/1078-0432; http://www.ama-assn.orq/resources/doc/usan/dabrafenib.pdf). Preferably the RAF inhibitor is sorafenib tosylate, Vemurafenib (also known as PLX4032, RG7204 or R05185426, e.g. marketed as Zelboraf, from Plexxikon (Daiichi Sankyo group) and Hoffmann-La Roche, or XL281 (Exelixis), or a derivative thereof. Preferably, the derivative of the BRAF inhibitor is a salt.

Other examples include those RAF kinase inhibitors, including B-RAF kinase inhibitors, disclosed in, for example, U.S. Pat. No. 6,987,119, WO98022103, WO99032436, WO2006084015, WO2006125101, WO2007027855, WO2005004864, WO2005028444, WO03082272, WO2005032548, WO2007030377, WO2010114928, WO2005123696, WO2007002325, US20090181371, WO2008120004, WO2006024834, WO2006067446, which patent applications can be referenced to the extent of their disclosure of RAF inhibitors, including B-RAF inhibitors and methods of making and using the same.

In particular examples, the RAF inhibitor is a small interfering nucleotide sequence capable of inhibiting RAF activity, such as siRNA using one or more small double stranded RNA molecules. For example, RAF activity in a cell can be decreased or knocked down by exposing (once or repeatedly) the cell to an effective amount of the appropriate small interfering nucleotide sequence. The skilled person knows how to design such small interfering nucleotide sequence, for example as described in handbooks such as Doran and Helliwell (RNA interference: methods for plants and animals Volume 10 CABI 2009).

A variety of techniques can be used to assess interference with RAF activity of such small interfering nucleotide sequence, such as described in WO 2005047542, for example by determining whether the candidate small interfering nucleotide sequence decreases BRAF activity. Candidate small interfering nucleotide sequences that are capable of interference may be selected to further analysis to determine whether they also inhibit proliferation of melanoma cells, for example by assessing whether changes associated with inhibition of proliferation of melanoma cells occurs in melanoma cells.

The RAF inhibitor according to the present invention may be a binding agent such as an antibody which specifically binds activated and/or mutated BRAF such as the ones described in WO 2005047542, or as described in US 20040096855.

A RAF inhibitor has RAF inhibitor activity, or in other words reduces activated (or mutated) RAF activity, which activity may be verified by method known to the skilled person, for example those disclosed in EP098638261.

A ERK polypeptide or peptide is to indicate a polypeptide having serine/threonine protein kinase activity, e.g. ERK phosphorylates and activates MAP (microtubule-associated proteins), and having at least 85% amino acid identity to the amino acid sequence of a human ERK, e.g to ERK1 (e.g. Gene accession number 5595; Refseq RNA Accessions NM_001040056.2; protein NP_001035145.1) or ERK2 (e.g. Gene accession number 5594; Refseq RNA Accessions NM_002745.4 ; protein NP_002736.3). The amino acid sequence of ERK enzymes, as well as other proteins mentioned herein, and variations thereof are available in GenBAnk, accessible via http://www.ncbi.nlm.nih.gov/genbank/ by entering either the numbers mentioned above or entering the relevant protein name.

By ERK biological activity is meant any function of ERK, such as enzymatic activity, kinase activity, the ability to phosphorylate an ERK substrate, or signaling the MAPK/ERK pathway.

By ERK inhibitor is meant a compound that reduces the biological activity of ERK; or that reduces the expression of an mRNA encoding an ERK polypeptide; or that reduces the expression of an ERK polypeptide. An ERK inhibitor can inhibit one member, several members or all members of the family of ERK kinases.

ERK (extracellular regulated kinase) is a group of MAP kinases which regulate the growth and proliferation of cells (Bokemeyer et al. 1996, Kidney Int. 49, 1187).

Embodiments of the invention include an ERK inhibitor that inhibits or reduces ERK protein expression, amount of ERK protein or level of ERK translation, amount of ERK transcript or level of ERK transcription, stability of ERK protein or ERK transcript, half-life of ERK protein or ERK transcript, prevents the proper localization of an ERK protein or transcript; reduces or inhibits the availability of ERK polypeptide, reduces or inhibits ERK activity; reduces or inhibits ERK, binds ERK protein, or inhibits or reduces the post-translational modification of ERK, including its phosphorylation. In analogy, the above described inhibitory action are also to be construed to apply, in comparable fashion to any inhibitor described herein for its specific target (e.g. a BRAF inhibitor for BRAF, etc.). In some embodiments the inhibitor is a selective inhibitor.

In some embodiments of the present invention, the ERK inhibitor is an ERK inhibitor such as disclosed in WO2002058687, for example SL-327 (Carr et al Psychopharmacology (Berl). 2009 Jan;201(4):495-5060). Further ERK inhibitors may be found in WO2002058687, AU2002248381, US20050159385, US2004102506, US2005090536, US2004048861, US20100004234, HR20110892, WO2011163330, TW200934775, EP2332922, WO2011041152, US2011038876, WO2009146034, HK1117159, WO2009026487, WO2008115890, US2009186379, WO2008055236, US2007232610, WO2007025090, and US2007049591. Reference is made to said documents with respect to their content regarding MEK inhibitors, and methods for making the same.

Further non-limiting examples or ERK-inhibitors include BVD-523, FR 180204 (CAS

No. 865362-74-9), Hypothemycin (CAS no. 76958-67-3), MK-8353, SCH9003531, Pluripotin (CAS no. 839707-37-8), SCH772984 (CAS no. 942183-80-4), and VX-11^{e} (Cas no. 896720-20-0).

In particular examples, the ERK inhibitor is a small interfering nucleotide sequence capable of inhibiting ERK activity, such as siRNA using one or more small double stranded RNA molecules. For example, ERK activity in a cell can be decreased or knocked down by exposing (once or repeatedly) the cell to an effective amount of the appropriate small interfering nucleotide sequence. The skilled person knows how to design such small interfering nucleotide sequence, for example as described in handbooks such as Doran and Helliwell (RNA interference: methods for plants and animals Volume 10 CABI 2009). Candidate small interfering nucleotide sequences that are capable of interference may be selected to further analysis to determine whether they also inhibit proliferation of melanoma cells, for example by assessing whether changes associated with inhibition of proliferation of melanoma cells occurs in melanoma cells.

The skilled person knows that analogues, derivatives or modified versions of the above-documented ERK inhibitors may be used in the context of the present invention, as long as such analogues, derivatives or modified versions have ERK inhibitor activity.

The ERK inhibitor according to the present invention may be a binding agent such as an antibody which specifically binds ERK, thereby inhibiting its function.

ERK inhibitor activity may be assayed in vitro, in vivo or in a cell line. In vitro assays include assays that determine inhibition of either the kinase activity or ATPase activity of activated ERK. Alternate in vitro assays quantitate the ability of the inhibitor to bind to ERK and may be measured either by radiolabelling the inhibitor prior to binding, isolating the inhibitor/ERK complex and determining the amount of radiolabel bound, or by running a competition experiment where new inhibitors are incubated with ERK bound to known radioligands. One may use any type or isoform of ERK, depending upon which ERK type or isoform is to be inhibited. An example of measuring ERK inhibitory activity is described in EP 1317453 B1.

A MEK polypeptide (e.g. Gene accession numbers 5604 or 5605; Refseq RNA Accessions NM_002755.3 or NM_030662.3; protein NP_002746.1 or NP_109587.1), protein or peptide is to indicate a polypeptide having serine/threonine protein kinase activity. For example MEK1 (e.g. Genbank Accession NO: NP002746) and MEK2 (e.g. Genbank Accession NO: NP109587) phosphorylates and activates MAPK. Another example is MEK3 ((e.g. Genbank Accession NO: NP002747). MEK comprises both MEK1 and MEK2: MAP/ERK kinase 1, MEK1, PRKMK1, MAPKK1, MAP2K1, MKK1 are the same enzyme, known as MEK1, MAP/ERK kinase 2, MEK2, PRKMK2, MAPKK2, MAP2K2, MKK2 are the same enzyme, known as MEK2. MEK1 and MEK2, together MEK, can phosphorylate serine, threonine and tyrosine residues in protein or peptide substrates. To date, few cellular substrates of MEK isoforms have been identified. The amino acid sequence of MEK enzymes, other proteins mentioned herein, and variations thereof are available in GenBAnk, accessible via http://www.ncbi.nlm.nih.gov/genbank/ by entering either the numbers mentioned above or entering the relevant protein name.

By MEK biological activity is meant any function of MEK, such as enzymatic activity, kinase activity, or signaling the MAPK/ERK pathway.

By MEK inhibitor is meant a compound that reduces the biological activity of MEK; or that reduces the expression of an mRNA encoding a MEK polypeptide; or that reduces the expression of a MEK polypeptide. A MEK inhibitor can inhibit one member, several members or all members of the family of MEK kinases. In one embodiment the MEK inhibitor is a selective inhibitor.

Preferred MEK inhibitors, already known in the art, include but are not limited to the MEK inhibitors PD184352 and PD98059, inhibitors of MEKI and MEK2 U0126 (see Favata, M., et al., Identification of a novel inhibitor of mitogen-activated protein kinase. J. Biol. Chem. 273, 18623, 1998) and SL327 (Carr et al Psychopharmacology (Berl). 2009 Jan;201(4):495-506), and those MEK inhibitors discussed in Davies et al (2000) (Davies et al Biochem J. 351, 95-105). In particular, PDI 84352 (Allen, Lee et al Seminars in Oncology, Oct. 2003, pp. 105-106, vol. 30) has been found to have a high degree of specificity and potency when compared to other known MEK inhibitors, and may thus be preferred. A preferred MEK inhibitor GSK1120212/Trametinib (GlaxxoSmithKline) has been approved for treatment of BRAF mutant melanoma under the name Mekinist. MEK162 (Novartis) is also preferred. Other MEK inhibitors and classes of MEK inhibitors are described in Zhang et al. (2000) Bioorganic & Medicinal Chemistry Letters; 10:2825-2828.

Further MEK inhibitors are for example described in Tecle et al Medicinal Chemistry Letters Volume 19, Issue 1, 1 January 2009, Pages 226-229; WO2009018238, WO2007/044084, WO2005/051300, WO2011/095807, WO2008124085, WO2009018233, WO2007113505, US2011105521, WO2011067356, WO2011067348, US2010004247, and

US2010130519. Reference is made to said documents with respect to their content regarding MEK inhibitors, and methods for making the same. GSK1120212 is an example of a further MEK inhibitor.

The MEK inhibitor may also preferably be selected from AZD6244, 4-(4-Bromo-2-fluorophenylamino)-N-(2-hydroxyethoxy)- 1 ,5-dimethyl-6-oxo- 1 ,6-dihydropyridazine-3-carboxamide or 2-(2-fluoro-4-iodophenylamino)-N-(2-hydroxyethoxy)- 1 ,5-dimethyl-6- oxo-I,6-dihydropyridine-3-carboxamide.

In another embodiment the MEK inhibitor is selected from 6-(4-Bromo-2-chloro-phenylamino)-7-fluoro-3-methyl-3H-benzoimidazole-5-carboxylic acid (2-hydroxy- ethoxy)-amide or a pharmaceutically acceptable salt thereof. In one embodiment the MEK inhibitor is 6-(4-Bromo-2-chloro-phenylamino)-7-fluoro-3-methyl-3H-benzoimidazole-5- carboxylic acid (2-hydroxy-ethoxy)-amide hydrogen sulphate salt. 6-(4-Bromo-2-chloro- phenylamino)-7-fluoro-3-methyl-3H-benzoimidazole-5-carboxylic acid (2-hydroxy- ethoxy)-amide hydrogen sulphate salt may be synthesized according to the process described in International Patent Publication Number WO2007/076245.

Furthermore, according to the invention the MEK inhibitor may be selected from the group consisting of certain experimental compounds, some of which are currently in Phase 1 or Phase II studies, namely PD-325901 (Phase 1, Pfizer), XL518 (Phase 1, Genentech), PD-184352 (Allen and Meyer Semin Oncol. 2003 Oct;30(5 Suppl 16):105-16.), PD-318088 (Tecle et al nic & Medicinal Chemistry Letters Volume 19, Issue 1, 1 January 2009, Pages 226-229), AZD6244 (Phase II, Dana Farber, AstraZeneca; WO2007/076245.) and CI-1040 (Lorusso et al Journal of clinical oncology 2005, vol. 23, no23, pp. 5281-5293).

Other examples of drugs that inhibit MEK include, PD-0325901 (Pfizer), AZD-8330 (AstraZeneca), RG-7167 (Roche/Chugai), RG-7304 (Roche), CIP-137401 (Cheminpharma), WX-554 (Wilex; UCB), SF-2626 (Semafore Pharmaceuticals Inc), RO-5068760 (F Hoffmann-La Roche AG), RO-4920506 (Roche), G-573 (Genentech) and G-894 (Genentech), N-acyl sulfonamide prodrug GSK-2091976A (GlaxoSmithKline), BI-847325 (Boehringer Ingelheim), WYE-130600 (Wyeth/Pfizer), ERK1-624, ERK1-2067, ERK1-23211, AD-GL0001 (ActinoDrug Pharmaceuticals GmbH), selumetinib (AZD6244), trametinib, TAK-733, Honokiol, MEK-162, derivates, and salts thereof.

In another embodiment the MEK inhibitor may inhibit (gene) expression of MEK, for example by interfering with mRNA stability or translation. In one embodiment the MEK inhibitor is selected from small interfering RNA (siRNA), which is sometimes known as short interfering RNA or silencing RNA, or short hairpin RNA (shRNA), which is sometimes known as small hairpin RNA. The skilled person knows how to design such small interfering nucleotide sequence, for example as described in handbooks such as Doran and Helliwell RNA interference: methods for plants and animals Volume 10 CABI 2009.

The MEK inhibitor according to the present invention may be a binding agent such as an antibody which specifically binds MEK, thereby inhibiting its function.

A number of assays for identifying kinase inhibitors, including MEK inhibitors, are known, for example from Downey et al. (1996) J Biol Chem.; 271(35): 21005- 21011 or EP2496575.

AXL (also known as UFO, ARK, and Tyro7; nucleotide accession numbers NM_021913 and NM_001699; protein accession numbers NP_068713 and NP_001690) is a receptor protein tyrosine kinase (RTK) that comprises a C-terminal extracellular ligand-binding domain and N-terminal cytoplasmic region containing the catalytic domain. Axl and its two close relatives, Mer /Nyk and Sky (Tyro3/Rse/Dtk), collectively known as the Tyro3 family of RTK's, all bind and are stimulated to varying degrees by the same ligand, Gas6 (growth arrest specific-6),a protein with significant homology to the coagulation cascade regulator, Protein S. One important function of AXL may be to mediate cell-cell adhesion. Axl is expressed in the vasculature in both endothelial cells (EC's) and vascular smooth muscle cells (VSMC's) and in cells of the myeloid lineage and is also detected in breast epithelial cells, chondrocytes, Sertoli cells and neurons.

The overexpression of AXL has been reported in a wide variety of solid tumor types including, but not limited to, breast, renal, endometrial, ovarian, thyroid, non-small cell lung carcinoma, and uveal melanoma as well as in myeloid leukemia's.

AXL inhibitors are known in the art and are for example described in AU2014200825, US2014065143, and EP2423208. These application also describe assay to determine AXL activity and/or inhibition, and are included herein by reference.

EGFR (epidermal growth factor receptor; OMIM: 131550 MGI: 95294 HomoloGene: 74545 ChEMBL: 203 GeneCards: EGFR Gene) inhibitors are also known in the art, and include including gefitinib, erlotinib, lapatinib and cetuximab. EGFR belongs to the HER family of receptor tyrosine kinases which are important mediators of cell growth, differentiation and survival. The receptor family includes four distinct members including epidermal growth factor receptor (EGFR, ErbB1, or HERI), HER2 (ErbB2 or p1 8 5 ″″), HER3 (ErbB3) and HER4 (ErbB4 or tyro2).

EGFR, encoded by the erbB1 gene, has been causally implicated in human malignancy. In particular, increased expression of EGFR has been observed in breast, bladder, lung, head, neck and stomach cancer as well as glioblastomas. EGFR kinase inhibitors, as for all inhibitors mentioned herein, include but are not limited to low molecular weight inhibitors, antibodies or antibody fragments, antisense constructs, small inhibitory RNAs (i. e. RNA interference by dsRNA; RNAi), and ribozymes. In a preferred embodiment, the EGFR kinase inhibitor is a small organic molecule or an antibody that binds specifically to the human EGFR.

EGFR kinase inhibitors that include, for example quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE 19629652. These application also describe assay to determine EGFR activity and/or inhibition, and are included herein by reference.

Platelet-derived growth factor receptors (PDGF-R) are cell surface tyrosine kinase receptors for members of the platelet-derived growth factor (PDGF) family. PDGF subunits -A and -B are important factors regulating cell proliferation, cellular differentiation, cell growth, development and many diseases including cancer. There are two forms of the PDGF-R, alpha and beta each encoded by a different gene.

Inhibitors for PDGFR and PDGFRbeta are well-known to the skilled person. Known inhibitors of PDGF-R tyrosine kinase activity includes quinoline-based inhibitors reported by Maguire et al. (J. Med. Chem. 1994,312129). and by Dolle et al. (.J. Med. Chem. 1994,37, 2627). A class of phenylamino-pyrimidine-based inhibitors was recently reported by Traxler et al. in EP 564409 and by Zimmerman, J.; and Traxler, P. et al. (Biorg. & Med. C'hem. Lett. 1996,6(11), 12211226) and by Buchdunger, E. et al. (Proc. ival. Acad. Sci., and for example described in EP1133480. Other inhibitors include low molecular weight inhibitors, antibodies or antibody fragments, antisense constructs, small inhibitory RNAs (i. e. RNA interference by dsRNA; RNAi), and ribozymes. In a preferred embodiment, the inhibitor is a small organic molecule or an antibody that binds specifically to the human PDGFR or PDGFRbeta.

Also inhibitors for IGF-IR (insulin-like growth factor 1 (IGF-1) receptor), EphhA7 (Ephrin type-A receptor 7 is a protein that in humans is encoded by the EPHA7 gene), EphA2 (Ephrin type-A receptor 2) and MER (Tyrosine-protein kinase Mer) are known to the skilled person and include low molecular weight inhibitors, antibodies or antibody fragments, antisense constructs, small inhibitory RNAs (i. e. RNA interference by dsRNA; RNAi), antibodies and ribozymes.

The current disclosure is based on on the surprising finding that a combination of an inhibitor of a protein of the MAPK/ERK pathway and an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL is advantageous in the treatment of cancer, preferably melanoma.

As will be understood by the skilled person, one or more inhibitor of one ore more proteins of the MAPK/ERK pathway, in combination with one or more inhibitors of one or more kinase selected from AXL, EGFR, PDGFR, IGF-IR, EphA7, PDGFRbeta, EphA2 and Mer may be used in the combinations according to the current disclosure.

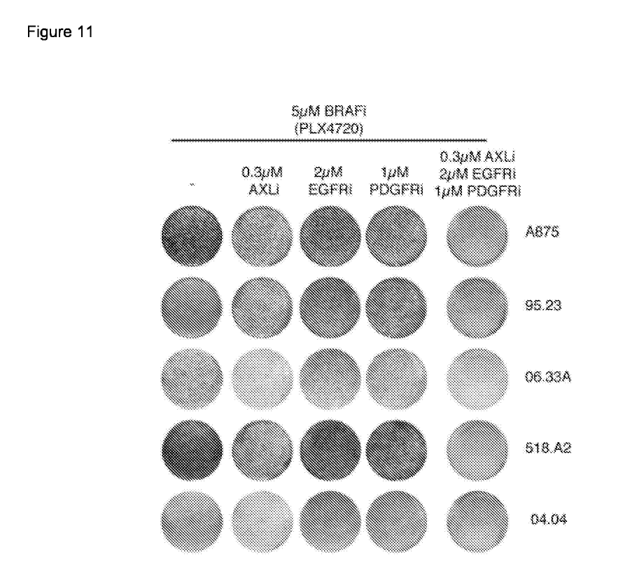

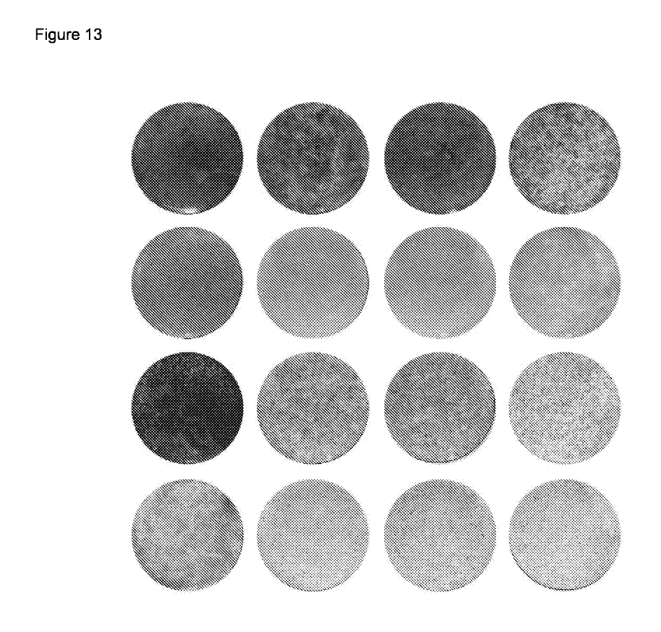

In a preferred embodiment, an inhibitor of a protein of the MAPK/ERK pathway and an inhibitor of AXL is combined. In another preferred embodiment, an inhibitor of a protein of the MAPK/ERK pathway and an inhibitor of AXL is combined with an inhibitor of a kinase selected from the group consisting of EGFR, PDGFR, IGF-IR, EphA7, PDGFRbeta, EphA2 and Mer. In this embodiment there is provided for a combination of an inhibitor of a protein of the MAPK/ERK pathway, an inhibitor of a AXL and an at least one inhibitor of a kinase selected from the group consisting of EGFR, PDGFR, IGF-IR, EphA7, PDGFRbeta, EphA2 and Mer, preferably EGFR and PDGFR for use as a medicament, preferably for use in the treatment of a cancer, preferably melanoma, in a patient. This combination can be used in the treatments according to the current disclosure. The combination may thus comprise three, four, five or more different inhibitors, wherein at least one is an inhibitor of a protein of the MAPK/ERK pathway, at least one is an inhibitor of AXL, and at least one is an inhibitor of at least one kinase selected from the group consisting of EGFR, PDGFR, IGF-IR, EphA7, PDGFRbeta, EphA2 and Mer, preferably EGFR and PDGFR. Obviously, the at least three inhibitors in this combination are not identical. It was surprisingly found that together with the relative changes in the expression of MITF and AXL, i.e. a reduced amount of MITF and increased amount of AXL, the expression of a kinase from the group consisting of EGFR, PDGFR, IGF-IR, EphA7, PDGFRbeta, EphA2 and Mer is altered, i.e. increased. It was likewise surprisingly found that by combining at least one an inhibitor of a protein of the MAPK/ERK pathway, at least one inhibitor of AXL, and at least one inhibitor of at least one kinase selected from the group consisting of EGFR, PDGFR, IGF-IR, EphA7, PDGFRbeta, EphA2 and Mer, preferably EGFR and PDGFR, dramatically further improves the treatment of the cancer, preferably cancer with acquired resistance to a protein of the MAPK/ERK pathway and/or a AXL (see the Examples, for example FIGS. 11 and 13. This may be even the case in those cells in which the expression of the at least one kinase selected from the group consisting of EGFR, PDGFR, IGF-IR, EphA7, PDGFRbeta, EphA2 and Mer, preferably EGFR and PDGFR has not detectably changed, i.e. wherein no overexpression has been shown. In particular the skilled person will understand that in a preferred embodiment the at least one inhibitor of at least one kinase selected from the group consisting of EGFR, PDGFR, IGF-IR, EphA7, PDGFRbeta, EphA2 and Mer, preferably EGFR and PDGFR is selected based on the expression of the corresponding kinase in the cancer cells to be treated. For example, a PDGFR inhibitor may be selected when the cells do overexpress PDGFR. The skilled person understands how, in this preferred embodiment, to define the patient group based on the expression of said kinase.

In a preferred embodiment of the current disclosure the cancer cells of the patients are characterized by low, or absent MITF expression, as shown in the examples. In this embodiment there is provided for the combinations, and used thereof, disclosed above, wherein the cancer in said patient is characterized by the absence of MITF protein, or by a reduced amount of MITF protein.

MITF, Microphthalmia-associated transcription factor (OMIM: 156845 MGI: 104554 HomoloGene: 4892 ChEMBL: 1741165 GeneCards: MITF Gene) is a transcription factor from the bHLH-LZ family which plays a major role in melanocyte survival and growth. MITF is involved in the regulation of melanogenesis. This factor is necessary for terminal melanocyte differentiation and/or pigmentation, on the one hand, and for malignant behavior by inducing cell proliferation, on the other hand. Constitutional “loss of function” mutations of the MITF gene are associated with autosomal dominant diseases such as Waardenburg syndrome and

Tietz syndrome, characterized by hearing loss and pigmentation anomalies of the skin, hair and/or iris.

The MITF gene comprises 9 exons. Six MITF isoforms have been identified. In humans they are generally referred to as isoforms 1 to 6, while isoform 4 is more commonly known as isoform M. In the mouse, the letter nomenclature is used instead. These isoforms are transcribed by specific promoters. In addition, they can be distinguished by their N-terminal region and all contain exons 2 to 9, whereas exon 1 is specific of each isoform. Isoform 4, more commonly known as MITF-M, has been detected in melanocytes or in vivo transformed cells (nevus, melanoma, etc.) or in vitro cell lines. The other isoforms are expressed in many tissues and cell lines, sometimes also with tissue specificities.

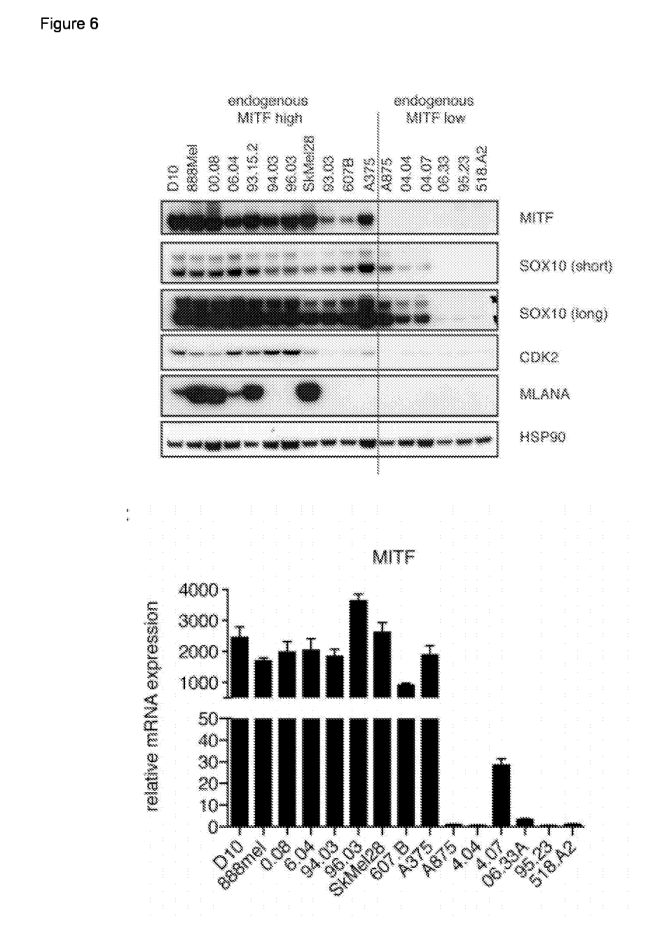

The skilled person knows how to determine MITF expression and whether MITF expression is reduced or even absent. For example, as can be witnessed from the examples, various tumours were found that exhibit strongly reduced expression of MITF in comparison to other tumours of the same type (e.g. melanoma), for example as can be witnessed by comparing relative mRNA expression (as shown in FIG. 6, lower panel) normalized to beta-actin as described in the Example. For this, the beta-actin is the expression to what is normalized. It is used as a housekeeping gene. The relative expression of MITF can be determined, for example, as follows: MITF expression is measured with a primerset among the samples and then one of the samples is arbitrary set at 1, in this case it is the A875 cell line. In comparison to this, for example, sample 04.07 expresses 30 times more MITF and others about 1000 times more. Thus cells expressing 5 times, preferably 10 times, more preferably 20, 30, 40 or 100 times less MITF as compared to those cells expressing 1000-fold more MITF compared to the A875 cell line, can be considered to have reduced or absent MITF expression. In other words, cells that express no more than 200 more MITF than the A875 cell line, normalized for beta-actin, may be considered the have reduced MITF or absent MITF expression.

In other words, reduced expression, absence of expression, overexpression and the like can be established by the skilled person by comparing the expression among various samples of the same sort (for example, amongst various melanoma's). Depending on, for example, the type of cancer, an increase or decrease of, for example 2-fold, 5-fold, 10-fold, 25-fold, 50-fold or 100-fold or more may be considered as “reduced” or “absent” or as “increased” amount (of expression) for a protein.

In another embodiment, there is provided for the combination disclosed above, wherein the cancer in said patient is characterized by the presence of a kinase selected from the group consisting of AXL, EGFR, PDGFR, IGF-IR, EphA7, PDGFRbeta, EphA2 and Mer, preferably AXL, EGFR and PDGFR, most preferably AXL, or by an increased amount of said kinase. As can be witnessed from the examples, it was found that, in particular in cancer cells with reduced or absent expression of MITF, this was accompanied by expression of, or increased amount of expression of a kinase selected from the group consisting of AXL, EGFR, PDGFR, IGF-IR, EphA7, PDGFRbeta, EphA2 and Mer. In particular it was found that AXL expression was inversely related to MITF expression. In other words, in cells with low or absent MITF expression, there was AXL expression, more in particular increased AXL expression.

Thus, in a preferred embodiment, the cancer to be treated in characterized by both a reduced amount of MITF expression and a increased amount of AXL expression, as can be witnessed from the examples. In such cancers, the ratio MITF-expression over AXL-expression is dramatically changed (e.g. 10-times, 20-times, 50-times, 100-times, 1000 times, or even more lower) in comparison to other tumours of the same type or sort (e.g. melanoma), as shown in the Examples.

Preferably, the amount of MITF protein and/or the kinase (AXL, EGFR, PDGFR,

IGF-IR, EphA7, PDGFRbeta, EphA2 or Mer) is determined using immuno-staining or by determining mRNA levels. This methods are well-known to the skilled person, as can be witnessed from the examples.

In a further embodiment of the current disclosure, the cancer is a BRAF-mutated cancer, a NRAS-mutated cancer or a KRAS-mutated cancer, preferably wherein the cancer is a BRAF-mutated melanoma, a NRAS-mutated melanoma or a KRAS-mutated melanoma. It was found that in particular in these types of mutated cancers, preferably melanoma, the combinations of the current disclosure can advantageously be used.

The term “RAS protein” as used herein means any protein which is a member of the ras-subfamily, a subfamily of GTPases involved in cellular signaling. As is known in the art, activation of RAS causes cell growth, differentiation and survival. RAS proteins include, but are not limited to, HRAS, KRAS and NRAS. The proteins differ significantly only in the C-terminal 40 amino acids.

These proteins are GTPases that function as molecular switches regulating pathways responsible for proliferation and cell survival. RAS proteins are normally tightly regulated by guanine nucleotide exchange factors (GEFs) promoting GDP dissociation and GTP binding and GTPase-activating proteins (GAPs) that stimulate the intrinsic GTPase activity of RAS to switch off signaling. Aberrant RAS function is associated with hyper-proliferative developmental disorders and cancer and in tumors is associated with a single mutation typically at codons 12, 13 or 61. A comprehensive overview of RAS mutations in cancer was reported by Prior et al (2012) Cancer Res; 2457-67.

The combination therapy disclosed herein is suitable for use in patients with KRAS-mutated (also referred to as or KRAS-mutant) cancer, and in a preferred embodiment particular useful in patients that are characterized by having a KRAS-mutant melanoma. The term “KRAS-mutated cancer”, and thus KRAS-mutated melanoma are well known to the skilled person. A comprehensive overview of RAS mutations, including KRAS-mutations, in cancer was reported by Prior et al (2012) Cancer Res; 2457 67. KRAS-mutant cells promote oncogenesis due to being mutationally activated, in most cases, at codon 12, 13 and 61. In total forty-four separate point mutations have been characterized in RAS isoforms, with 99.2% in codons 12, 13 and 61. The protein product of the normal KRAS gene performs an essential function in normal tissue signaling, and the mutation of a KRAS gene is an essential step in the development of many cancers.

The GTPase KRAS, also known as V-Ki-ras2 Kirsten rat sarcoma viral oncogene homolog or KRAS, is a protein that in humans is encoded by the KRAS gene (e.g. Gene accession number 3845; Refseq RNA Accessions NM_004985.3; protein NP_004976.2). Like other members of the Ras family, the KRAS protein is a GTPase and is an early player in many signal transduction pathways. KRAS acts as a molecular on/off switch. Once it is turned on it recruits and activates proteins necessary for the propagation of growth factor and other receptors signal, such as c-Raff and PI 3-kinase.

In a preferred embodiment, the combination therapy disclosed herein is for use in patients with NRAS-mutated (also referred to as or NRAS-mutant) cancer, and in a preferred embodiment particular useful in patients that are characterized by having a NRAS-mutated melanoma. The term “NRAS-mutated cancer” and therefor NRAS-mutated melanoma are well known to the skilled person. A comprehensive overview of RAS mutations, including NRAS-mutations, in cancer was reported by Prior et al (2012) Cancer Res; 2457-67. NRAS-mutant cells promote ontogenesis due to being mutationally activated, in most cases, again at codon 12,13 and 61.

The NRAS protein is a GTPase enzyme that in humans is encoded by NRAS (neuroblastoma RAS viral (v-ras) oncogene homolog) gene gene (e.g. Gene accession number 4893; Refseq RNA Accessions NM_002524.4; protein NP_002515.1). The N-ras gene specifies two main transcripts of 2 Kb and 4.3 Kb, both transcripts appear to encode identical proteins as they differ only in the 3 untranslated region.

The combination therapy disclosed herein is suitable for use in patients with BRAF-mutated (also referred to as or BRAF-mutant) cancer, and in a preferred embodiment particular useful in patients that are characterized by having a BRAF-mutant melanoma. The term “BRAF-mutated cancer” and therefor BRAF-mutated melanoma are well known to the skilled person. BRAF (e.g. Gene accession number 673; Refseq RNA Accessions NM_004333.4 ; protein NP_004324.2) , is a member of the RAF family, which includes ARAF and CRAF in humans (Ikawa, Mol Cell Biol. 8(6):2651-4 (1988)). BRAF is a serine/threonine protein kinase and participates in the RAS/RAF/MEK/ERK mitogen activated protein kinase pathway (MAPK pathway, see Williams & Roberts, Cancer Metastasis Rev. 13(1):105-16 (1994); Fecher et al 2008 Curr Opin Oncol 20, 183-189 or Cargnello M, Roux PP. Microbiol Mol Biol Rev. 2011 Mar;75(1):50-83). Approximately 40-60% of (cutaneous) melanomas carry a mutation in the BRAF protein. Approximately 90% of these mutations result in the substitution of glutamic acid for valine at codon 600 (BRAF V600E, although other mutations are also known (e.g. BRAF V600K and BRAF V600R). Such mutation in BRAF typically leads to proliferation and survival of melanoma cells (Davies et al Nature 2002; 417:949-54; Curtin et al N Engl J Med 2005;353:2135-47), through activation of the MAPK/ERK pathway. As is well-known to the skilled person, this pathway plays a significant role in modulating cellular responses to extracellular stimuli, particularly in response to growth factors, and the pathway controls cellular events including cell proliferation, cell-cycle arrest, terminal differentiation and apoptosis (Peyssonnaux et al., Biol Cell. 93(I-2):53-62 (2001)).

The amino acid sequence of BRAF, NRAS or KRAS protein and any other protein mentioned herein, and variations thereof are available in GenBAnk, accessible via http://www.ncbi.nlm.nih.gov/genbank/.

As already discussed in detail above, in an embodiment, there is provided that said inhibitor of a protein of the MAPK/ERK pathway is selected from the group consisting of a RAF-inhibitor, an ERK-inhibitor, and a MEK-inhibitor. Various inhibitors directed to RAF, e.g. B-RAF, ERK and or MEK are known to the skilled person.

In a preferred embodiment, the inhibitor of the kinase is an inhibitor of AXL. As discussed above, even more preferably, there is provided the combination of an inhibitor of a protein of the MAPK/ERK pathway, an inhibitor of AXL and an inhibitor of a kinase selected from the group consisting of EGFR, PDGFR, IGF-IR, EphA7, PDGFRbeta, EphA2 and Mer, preferably EGFR and PDGFR, for use in treatment of cancer, in particular melanoma, for example characterized by reduced or absent MITF expression and/or increased AXL expression.

According there is provided for an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor, for use in treatment of a cancer, preferably melanoma, in a patient, wherein said inhibitor of a protein of the MAPK/ERK pathway is administrated simultaneously, separately or sequentially with an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL.

Also provided is an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL, for use in treatment of a cancer, preferably melanoma, in a patient, wherein said inhibitor of the kinase is administrated simultaneously, separately or sequentially with an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor.

These inhibitors for use in treatment of a cancer in a patient as disclosed above are preferably for use, wherein the cancer in said patient is characterized by the absence of MITF protein or by a reduced amount of MITF protein. This may be compared to a certain threshold value in patients with the same type of cancer (e.g. melanoma), using techniques well-known to the skilled person.

The inhibitor for use in treatment of a cancer in a patient according as disclosed above are preferably for use, in a cancer as disclosed above, including a cancer characterized by absence of MITF protein or by a reduced amount of MITF protein, wherein the cancer in said patient is characterized by the presence of a kinase selected from the group consisting of AXL, EGFR, PDGFR, IGF-IR, EphA7, PDGFRbeta, EphA2 and Mer, preferably AXL, EGFR and PDGFR, most preferably AXL, or by an increased amount of said kinase. This may be compared to a certain threshold value in patients with the same type of cancer (e.g. melanoma), using techniques well-known to the skilled person.

Also provided is a product, preferably a pharmaceutical product, comprising an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor, and an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL, as a combined preparation for simultaneous, separate or sequential use in treatment of a cancer, preferably melanoma, in a patient, and as disclosed above.

Also provided is a method for the treatment of a cancer, preferably melanoma, in a patient, wherein the method comprises the simultaneous, separate or sequential administering to the patient of an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor, and an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL.

In a preferred embodiment, the method comprises the simultaneous, separate or sequential administering to the patient of an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor, an inhibitor of AXL and an inhibitor of a kinase selected from the group consisting of EGFR, PDGFR, IGF-IR, EphA7, PDGFRbeta, EphA2 and Mer, preferably EGFR and PDGFR. Even more preferred, an inhibitor of a protein of the MAPK/ERK pathway, an inhibitor of AXL, an inhibitor of EGFR and an inhibitor of PDGFR is used.

Preferably, the cancer in said patient is characterized by the absence of MITF protein or by a reduced amount of MITF protein Preferably, the cancer is a BRAF-mutated cancer, a NRAS-mutated cancer or a KRAS-mutated cancer, preferably wherein the cancer is a BRAF-mutated melanoma, a NRAS-mutated melanoma or a KRAS-mutated melanoma.

Also provided is a method for the diagnosis of cancer of a patient, the method comprising the steps of determining the level of MITF in the cancer cell(s) obtained form said patient; and determining the level of a kinase and/or the phosphorylation status selected from the group consisting of AXL, EGFR, PDGFR, IGF-IR, EphA7, PDGFRbeta, EphA2 and Mer, preferably AXL, EGFR and PDGFR, most preferably AXL, in the cancer cell(s) obtained from said patient.

In a preferred embodiment the method for diagnosis comprises the step of determining whether the cancer cell(s) obtained from said patient are BRAF-mutated cancer, a NRAS-mutated cancer or KRAS-mutated cancer cells.

Also provided is a method for predicting treatment response of a cancer, preferably melanoma, of a patient, the method comprising the step of determining the amount of MITF in cancer cell(s) obtained form said patient; and determining a predicted treatment response based on the determined amount of MITF, wherein the absence of MITF protein or a reduced amount of MITF indicates a bad predicted treatment response. In a preferred embodiment, the above method further comprises the determining of the amount of AXL, EGFR, PDGFR, IGF-IR, EphA7, PDGFRbeta, EphA2 and/or Mer, preferably AXL expression, wherein increased expression thereof, together with the absence of MITF protein or a reduced amount of MITF indicates a bad predicted treatment response.

The treatment for which the response is predicted is preferably treatment only using one or more inhibitors of the MAPK/ERK pathway.

The method for predicting treatment response is preferably performed in vitro, i.e. outside the body, e.g the human body.

### EXAMPLES

### Example 1

Introduction

The mechanistic basis for intrinsic drug resistance in cancers like melanoma, in particular in BRAF mutant melanoma patients is elusive. Consistent with previous findings, we find that increased expression of the melanocyte master switch MITF protects against BRAF inhibition. But unexpectedly, the absence of MITF was associated with more severe intrinsic and acquired resistance to many targeted inhibitors in vitro and in vivo. Indeed, the presence of endogenous MITF was essential for adequate drug responses. MITF loss correlated with high levels of several receptor tyrosine kinases, particularly AXL. Inhibition of these receptor tyrosine kinases like AXL cooperated with BRAF inhibition in eliminating melanoma cells. Our results demonstrate that absence of MITF predicts multidrug resistance, and suggests the combinatorial targeting of BRAF signalling and these receptor kinase kinases, in particular AXL, in MITF-low BRAF mutant cancers melanomas. Likewise the data suggests the such combinatorial targeting in NRAS or KRAS mutated cancers, including NRAS or KRAS mutated melanoma.

Recently an ERK inhibitor (SCH772984) with a dual mechanism of action was described. It inhibits the enzymatic activity of ERK and its phosphorylation by MEK (Morris et al. Cancer Discov. (2013);3(7):742-50). This inhibitor can effectively block proliferation of BRAF and BRAF/MEK inhibitor-resistant cells and was therefore proposed as a new line of treatment on BRAF mutant (resistant) melanoma.

We performed a gain-of-function screen using the validation-based insertional-mutagenesis system (VBIM) (Lu et al. Proc Natl Acad Sci U S A. (2009) 22;106(38):16339-44) for factors mediating resistance to the aforementioned ERK inhibitor. We identified an insertion in the MITF (Microphthalmia-associated transcription factor) locus, resulting in an upregulation of the master lineage transcription factor MITF which is responsible for pigmentation and indispensible for the development of the melanocytic lineage. MITF expression is usually maintained in melanoma and was found to be amplified in 15% of metastatic melanoma suggestive of its oncogenic role Garraway, Nature.

Strikingly, in addition to gain of MITF expression, we also find acquired resistance strongly accompanied by loss of MITF expression and its target gene products in vitro and in vivo. In addition we find that in treatment nave melanoma cells very low levels or absence of MITF was invariably associated with intrinsic resistance to inhibitors acting across the BRAF/MEK/ERK pathway, including combinations, in vitro. Mechanistically, in nave MITF low cells we found a remarkable inverse correlation between absence of MITF and increased expression levels of the receptor tyrosine kinases EGFR, PDGFRbeta and AXL. In addition we detect a striking inverse correlation between MITF loss in acquired resistance and gain of AXL expression, suggesting that the receptor tyrosine kinase protects BRAF mutant melanoma cells from targeted therapy and that its expression is, either direct or indirect, negatively regulated by MITF.

However, as combinatorial treatment with drugs targeting the BRAF/MEK/ERK pathway and EGFR inhibitors failed to kill intrinsic and acquired resistant melanoma cells MAPK-pathway inhibition combined with EGFR and AXL inhibition reduced cell growth significantly in innate and acquired resistance.

### Materials and Methods

Validation-based insertional mutagenesis screen:

The insertional mutagenesis screen was performed as described previously (Lu et al. Proc Natl Acad Sci U S A. (2009) 22;106(38):16339-44). Briefly, an intermediate sensitive melanoma cell line of low passage (Me104.07) was infected separately with the three different VBIM constructs (SD1-3). Two days after infection cells were exposed to 1 μM 50H772984 for three weeks till single colonies had formed. These were picked and separately cultured. For identification of insertions Splinkerette-PCR was used according to the published protocol and Sanger sequencing applied on amplified products.

Vectors:

Human MITF-M was amplified from human melanocyte cDNA and cloned into pcDH puro using EcoR1 and Not1 restriction sites.
Forward primer used was:

GGGGAATTCATGGATGCTGGAAATGCTAGAATATAATCACTATCAGG

Reverse primer used was:

GGGGCGGCCGCCTAACAAGTGTGCTCCGTCTCTTCC

shRNAs in pLKO puro were picked from the TRC library. Sh Luciferase was used as a control.

MITF#19:
CCGGCCAACTTCTTTCATCAGGAAACTCGAGTTTCCTGATGAAAGAAGTT
GGTTTTT
MITF#20:
CCGGCGGCATTTGTTGCTCAGAATACTCGAGTATTCTGAGCAACAAATGC
CGTTTTT
MITF#22:
CCGGCGTGGACTATATCCGAAAGTTCTCGAGAACTTTCGGATATAGTCCA
CGTTTTT
MITF#23:
CCGGCGGGAAACTTGATTGATCTTTCTCGAGAAAGATCAATCAAGTTTCC
CGTTTTT

qRT-PCR and Primers

RNA was isolated using Trizol (Ambion), following standard protocol. Reverse transcription was performed using a kit from Invitrogen. Primers were designed using Primer Express software. QRT-PCR was performed with SYBR green master mix (Applied Biosystems) on a StepOnePlus Real-Time PCR system (Applied Biosystems, Warrington, UK). RNA levels were normalized against beta-actin or RPL13.
Following primers were used:

| | |
|---|---|
| MITF forward: | CAGGCATGAACACACATTCAC. |
| MITF reverse: | TCCATCAAGCCCAAGATTTC. |
| Beta actin forward: | CCAACCGCGAGAAGATGA. |
| Beta actin reverse: | CCAGAGGCGTACAGGATAG. |
| RPL13 forward: | [symbol] |
| RPL13 reverse: | [symbol] |

[symbol]

indicates data missing or illegible when filed

### Inhibitors and Solvents

MEK inhibitor GSK1120212/Trametinib, BRaf inhibitors PLX-4720 and GSK211436/Dabrafenib, EGFR inhibitor Gefitinib and c-Kit/PDGFR inhibitor lmatinib/Gleevec were all purchased from Selleck Chemicals (Houston, Tex., USA). ERK inhibitor 5CH772984 was provided by Merck & Co, Whitehouse Station, NJ, USA (via a MTA). AXL inhibitor R428 from Axon Medchem. (Groningen, the Netherlands), the metabolic poison phenyl arsine oxide
(PAO) and solvent dimethylsulfoxide (DMSO) from Sigma-Aldrich (St. Louis, Mo., USA). Vemurafenib was commercially purchased (Selleck Chemicals, Houston, Tex.). All drugs were reconstituted in 100% dimethyl sulfoxide (DMSO) to a final concentration of 10 mM.

Cell lines, cell culture conditions and inhibitor treatments:

Cell line sources:
Melanoma cell lines and HEK293T were cultured in DMEM/9% FBS (Sigma), 2 mM glutamine, 100 U/mL penicillin and 0.1 mg/mL streptomycin (all Gibco) under standard conditions. HEK293T cells were used for virus production for MITF-overexpression and shRNAs. Briefly HEK293T cells were transfected with the plasmid of interest and the helper plasmids (pMDLglpRRE, pHCMV-G, and pRSVrev). Viral supernatant was either fresh frozen or subsequently used for infection. Infected melanoma cells were positively selected with puromycin (Sigma).
For dose response curve and colony formation assay cells were plated in a line-specific manner. Equal concentration of inhibitors was added one day after set up. For short-term viability assay (dose response curve) cells were plated in a 96-well format and drugs diluted with the HP D300 Digital Dispenser (Tecan). After three to five days incubation old medium was replaced by a dilution of CellTiter Blue reagent or CellTiter-Glo Luminescent Cell Viability Assay (Promega, Madison, Wiss.) in medium. Two hours later luminescence was measured by the infinite M200 microplate reader (Tecan, Giessen, Germany).
Long-term viability assays were performed in 6-well or 12-well format. Inhibitors solutions were replaced every two to three days and plates stained with crystal violet after six to nine days of treatment (as indicated). For immunoblot analysis cells were treated on 10 cm dishes and snap-frozen after harvesting.

Invasion assay and P-RTK array:

To examine the invasive properties 200.000 freshly trypsinized cells were seeded on Matrigel coated chambers (BD Biosciences) in serum-free medium. The lower compartment contained medium supplemented with 9% FCS. After overnight incubation non-invasive cells were removed from the chamber and invaded cells were stained with crystal violet. Pictures of invaded cells were taken on an Axio Vert Al microscope (Zeiss).
Phospho-RTK assay was performed using the human Phospho-RTK Array from RD Systems (Minneapolis, USA). All steps were performed according to the manufactures protocol.

Immunohistochemistry:

EAF-fixed tumor samples were embedded in paraffin and stained with Hematoxilin/Eosin according to common procedures.

Antibodies:

Cells were lysed in RIPA buffer and protein concentration measured with Bio-Rad protein assay. Immunoblots were performed according to standard protocols on 4 to 12% bis-Tris precast or 3-8% tris-acetate gels (NuPage).
The following antibodies were used:
Phospho-Akt (sc-7985-R), Axl (sc-1096) ,Bcl2 (sc-492), Cdk2 (sc-163), Cdk4 (sc-601) and EGFR (sc-03) were purchased from Santa Cruz. E-Cadherin (610181) and N-cadherin (610920) from BD Biosciences. MAP kinase p44/42 thy202/Tyr204 (9106) and PDGFRbeta (31695) from Cell Signaling. MelanA (MS_716P0) and MITF (MS-771-P1) from Neomarkers. MITF (ab12039) and Sox10 (ab-17732) from Abcam

RNA-seq analysis:

IIlumina 50 bp paired-end RNAseq data was collected on a panel of melanoma cell lines. Read mapping was performed using TopHat version 2.0.9 with the NCB! Build 37 reference genome {Trapne11:2009dp}. Read counts per gene were quantified using HTSeq version 0.5.4. Counts were adjusted for gene length and GC content and quantile normalized using the CON R package to obtain gene level offsets (Hansen 2011). Read counts were fitted to a generalized linear model with offsets for the final normalization step using the DESeq2 R package {Anders:2010fu}. Pearson correlation coefficients between genes for all samples were calculated using R.

‘Our’ analysis:

Paired-end 90 bp raw reads as generated by the IIlumina HiSeq 2000 were aligned to hg19 Sanger reference using TopHat (2.0.9) and bowtie2 (2.1.0). HTSeq (v.0.5.4) was used to generate the count matrix with the Ensembl GTF file (Homo_sapiens.GRCh37.74.gtf). Heatmaps were generated with DESeq (1.12.1) and gplots (2.12.1) as available through Bioconductor. In DESeq the dispersion estimate estimateDispersions had parameters:
‘method=“per-condition”’ and ‘fitType=“local”’ and for null model evaluation with no replicates ‘method=“blind”’, ‘fitType=“local”’ and ‘sharingMode=“fit-only”.
Analysis was performed, and plots were made using the statistical programming language R (v 3.0.2).

RESULTS

An Insertional Mutapenesis Screen Identifies MITF Overexpression as a Driver of MAPK-Pathway Inhibitor Resistance

To identify proteins conferring resistance to MAPK pathway inhibition by the recently available ERK inhibitor 5CH772984, we performed a lentiviral insertional mutagenesis screen using the VBIM system. Carrying a GFP-sequence and a strong CMV promoter, the virus integrates in the genome, resulting in an activation of downstream sequences that leads to overexpression of a FLAG-tagged protein. Due to Ore-mediated excision the insertion can be excised by addition of 4-Hydroxytamoxifen (4-OHT). We used a low passage human BRAFV600E mutant melanoma cell line (04.07), which is intermediately sensitive to the inhibitor (not all cells are killed by the ERK inhibitor even when used at high concentration). We infected this cell line with the three versions of the insertional mutagenesis vector (SD1-3). After three weeks of culturing in the presence of 1 μM SCH772984, we were able to pick drug-resistant clones and individually expanded them. Resistance occurred due to an advantageous insertion, as resistance could be reverted by 4-OHT-treatment (data not shown). In the majority of the clones, we detected a Flag-tagged protein, indicating that a successful in-frame insertion had occurred into the genome, leading to overexpression of a fusion protein. We observed an approximately 55 kDa FLAG-tagged protein in multiple independent clones, raising the possibility that a common gene was activated. Using splinkerette PCR followed by sequencing we identified an insertion in intron 2-3 of the MITF gene locus (data not shown). This finding could be confirmed by qPCR and immunoblotting of six individual clones.

To validate these results, we overexpressed MITF using a lentiviral system. Exogenous overexpression of MITF promoted survival and proliferation in three independent BRAFV600E melanoma cells lines after ERKi treatment. This was observed in spite of complete ERK pathway inhibition. The same protective effect of MITF was seen for PLX4720 or a MEK inhibitor (GSK1120212).

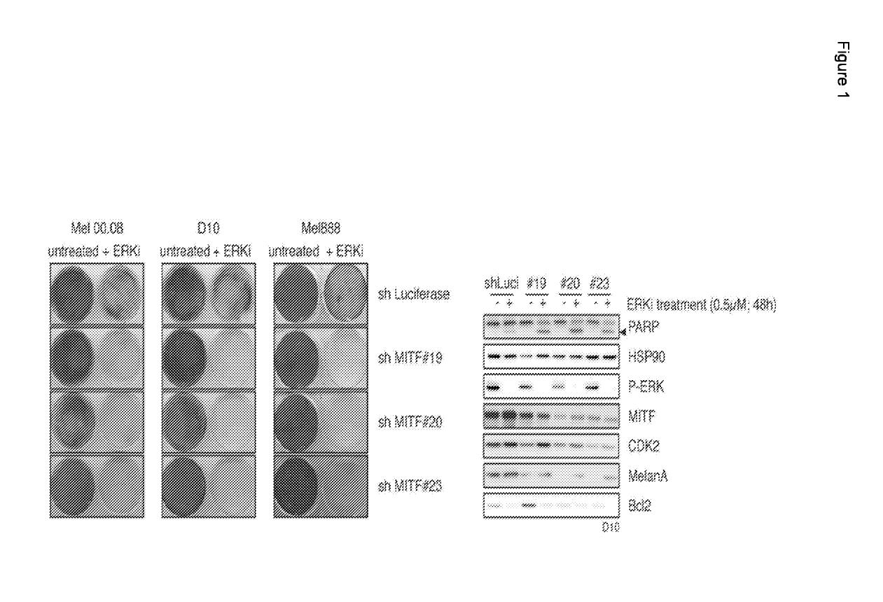

Having demonstrated that ectopic overexpression of MITF is sufficient to drive resistance to MAPK pathway inhibition, we next investigated whether conversely, depletion of MITF sensitizes melanoma cells to ERK inhibition. MITF was knocked down using three independent lentiviral shRNAs in three different BRAFV600E melanoma cell lines with high endogenous MITF expression. Efficient silencing of MITF, which caused downregulation of its target gene products Cdk2, Bcl2 and MelanA, resulted in sensitization to ERK inhibition, as demonstrated by colony formation assays and PARP cleavage (FIG. 1). Together, these data indicate that while exogenous overexpression of MITF drives resistance to MAPK pathway inhibition, maintenance of high endogenous MITF levels is required to protect from pathway inhibition. These observations are in agreement with, and extend, recent findings on the role of MITF in the response a MEK and ERK inhibitor.

MITF Expression is Frequently Lost in Acquired Resistance in Vitro and in Vivo

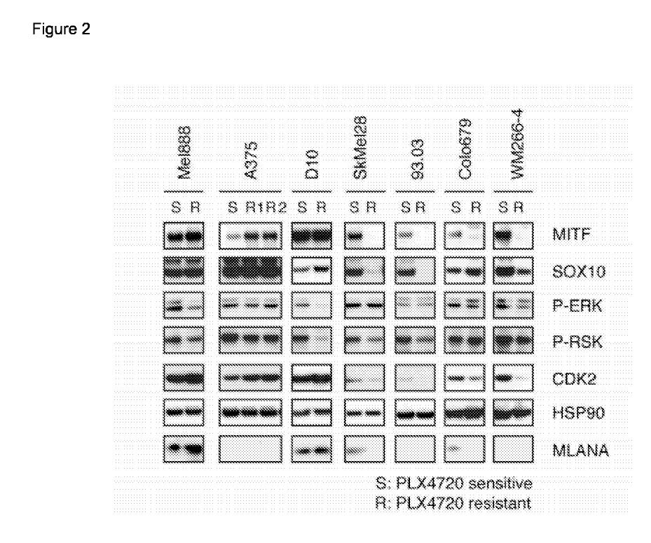

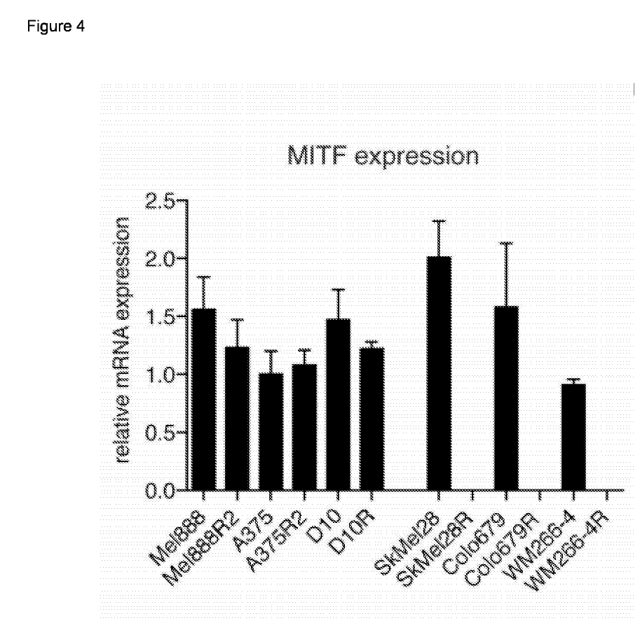

To determine the regulation of MITF expression during acquired resistance, we made several cell lines resistant to 1 μM SCH772984 or 3μM PLX4720 by increasing drug exposure. In line with our findings from the VBIM screen, we observed a moderate increase in MITF expression in three out of seven PLX4720-resistant lines (FIG. 2; Me1888, A375, D10), along with increased expression of several MITF targets and pigmentation of the cells. In contrast, four out of seven resistant lines (Skme128, Colo679, WM266-4 and 93.03) had downregulated MITF expression to almost undetectable levels relative to their parental counterparts. In almost all resistant cell lines ERK and RSK were reactivated to the level of their untreated counterparts. Similarly, four out of six cell lines resistant to 1 μM SCH772984 had decreased expression of MITF. Loss of MITF was followed by decreased expression of several target genes. The loss of MITF occurs on the transcriptional level, as qPCR analysis show almost undetectable levels of MITF expression (FIG. 4). Thus, while one set of drug-resistant cell lines upregulated MITF expression; we call these MITF^{acq}^{_}^{gain} cells. In contrast, for at least half of the cell lines we observed the exact opposite: a sharp downregulation of MITF expression; we call these MITF^{acq}^{_}^{loss} cells. The differential regulation of MITF during the acquisition of drug resistance may indicate that these two sets of cell lines, which both express MITF, are differently wired.

Therefore, we next investigated whether MITF^{acq}^{_}^{gain} and MITF^{acq}^{_}^{loss} cells show different drug responses upon MITF depletion, too. Indeed, D10 MITF^{acq}^{_}^{loss} cells showed an increased response to BRAF inhibition upon MITF depletion, as judged by reduced colony formation and induction of PARP cleavage. In contrast, SkMel28 MITF^{acq}^{_}^{loss} cells were not sensitized by MITF depletion. These results support the idea that, indeed, there are two distinct classes of MITF-expressing melanoma cell lines, which differ in their response to MAPK pathway inhibition: in MITF^{acq}^{_}^{gain} cells, MITF signaling is activated and contributes to drug resistance. In MITF^{acq}^{_}^{loss} cells, in which MITF is downregulated MITF expression is not required for the drug response.

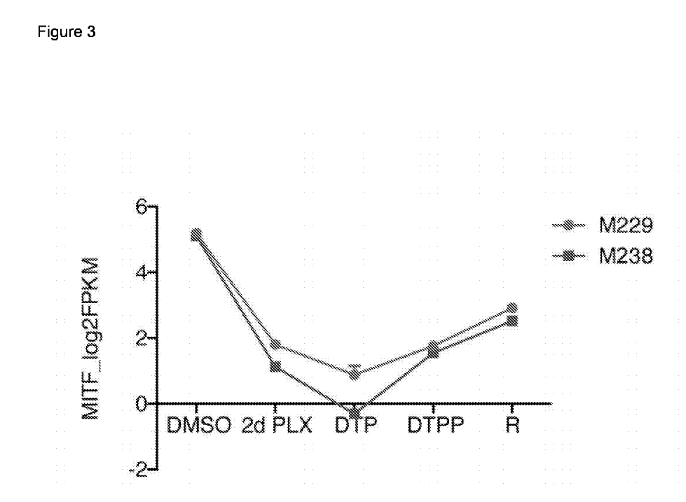

To study the dynamics of MITF downregulation as a function of the acquisition of a resistant phenotype, two independent MITF^{acq}^{_}^{loss} melanoma cell lines (M229 and M238; {Shi:2014fm}) were permanently exposed to 1 μM vemurafenib and monitored in time. MITF expression sharply dropped upon short-term exposure to PLX4720 and was further decreased in the remaining drug-tolerant population (DTP), drug-tolerant proliferating population (DTPP) and in resistant (R) cells (FIG. 3). These results show that acquired resistance is accompanied by a rapid decrease in MITF, and confirm that this is regulated at the transcriptional level.

To corroborate these in vitro findings in a physiologically more relevant setting, we performed immunohistochemical staining for MITF on human melanoma biopsies. The samples were taken from the patients before treatment and after patient relapsed with vemurafenib-resistant tumours. In one out of four (pre-and post-treatment) sample sets, we observed a dramatic drop in MITF abundance. For another patient, we observed a remarkable differential response: while there was abundant expression in the pre-treatment sample, one relapsed melanoma showed elevated levels of MITF, whereas another had lost detectable expression in most of the resistant cells. These results are in agreement with our findings in cultured melanoma cells and indicate that melanoma drug resistance in vivo is associated not only with gain of MITF expression but also with loss, even in different relapsed tumour clones from the same patient.

To validate these observations in a larger and independent set of melanomas, we determined MITF mRNA levels in a series of human BRAF^{V600E} mutant melanoma samples comparing them before and after treatment, when resistance had occurred. Although to a lesser extend than gain of MITF, we also find MITF lost in the resistant tumours compared to their treatment-naïve counterparts. Again, in the same patient, MITF can either be lost or gained in different biopsies due to resistance. Thus, MITF^{acq}^{_}^{gain} and MITF^{acq}^{_}^{loss} cells co-exist in vivo and the loss of MITF is frequently seen in the context of MAPK pathway inhibition.

MITF^{acq}^{_}^{loss} Cells are Cross-Resistant to Pathway Inhibition and Highly Invasive

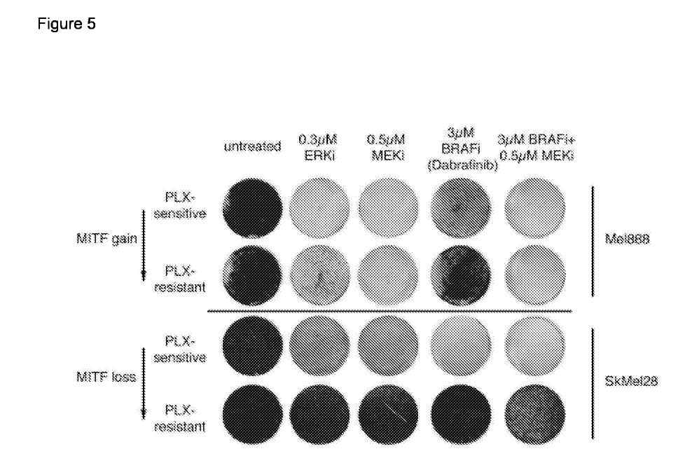

To characterize the differences between the MITF^{acq}^{_}^{gain} and MITF^{acq}^{_}^{loss} cells, we exposed them to several inhibitors of the MAPK-pathway, either alone or in combination. PLX4720-resistant MITF^{acq}^{_}^{gain} cells were resistant to another BRAF inhibitor (Dabrafenib) but were as sensitive to MEK or ERK inhibition as their drug-sensitive counterparts in a long-term experiment (FIG. 5). In contrast, PLX4720-resistant MITF^{acq}^{_}^{loss} cells displayed cross-resistance to the full panel of MAPK-pathway inhibitors, even when they were used in combination (FIG. 5). Phosphorylation of ERK was similarly suppressed across all cell lines, demonstrating that in all cases the drugs were equally effective to their respective targets. By contrast, only PLX4720-resistant MITF^{acq}^{_}^{gain} cells underwent apoptosis (as judged by PARP cleavage), whereas MITF^{acq}^{_}^{loss} cells did not upon any drug treatment. In a larger set of melanoma cell lines, comparing three MITF^{acq}^{_}^{gain} and three MITF^{acq}^{_}^{loss} cell lines, we observed that the latter tolerate exposure to higher drug concentrations than MITF^{acq}^{_}^{gain} cells, and hence are more prone to develop cross-resistance.

In melanoma, low MITF expression is known to be associated with a phenotypic switch, including increased invasiveness. To determine whether MITF^{acq}^{_}^{loss} cells show such properties, we transferred them to a matrigel-coated chamber and monitored their invasive potential. MITF^{acq}^{_}^{loss} cells had increased invasive capacity relative to MITF^{acq}^{_}^{loss} cells. In MITF^{acq}^{_}^{loss} cell lines in which E-cadherin was expressed, this epithelial protein was lost upon the acquisition of drug resistance. In addition the transcription factor Fra1, known to be involved in invasion and metastasis, is upregulated in all three MITF^{acq}^{_}^{loss} cells. Thus, in addition to showing a more cross-resistant phenotype than MITF^{acq}^{_}^{gain} cells, MITF^{acq}^{_}^{loss} cells are much more invasive.

Cells with Low Endogenous MITF Expression are Intrinsically Insensitive to MAPK Pathway Inhibition.

The above experiments focused on the regulation and role of MITF in the context of acquired targeted drug resistance. Next, we investigated the impact of endogenous MITF expression on treatment-naïve melanoma cells regarding sensitivity to MAPK pathway inhibition. Therefore, we grouped BRAF mutant melanoma cells in “MITF^{endo}^{_}^{hi}” and “MITF^{endo}^{_}^{lo}” cells based on their protein expression (FIG. 6 and MITF-specific transcript expression (FIG. 6). As expected, most MITF^{endo}^{_}^{hi} cell lines expressed MITF target gene products like CDK2 and MelanA, whereas those proteins were lower or even undetectable in MITF^{endo}^{_}^{lo} cells.

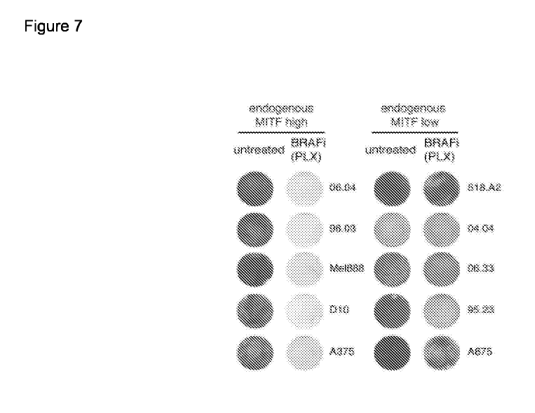

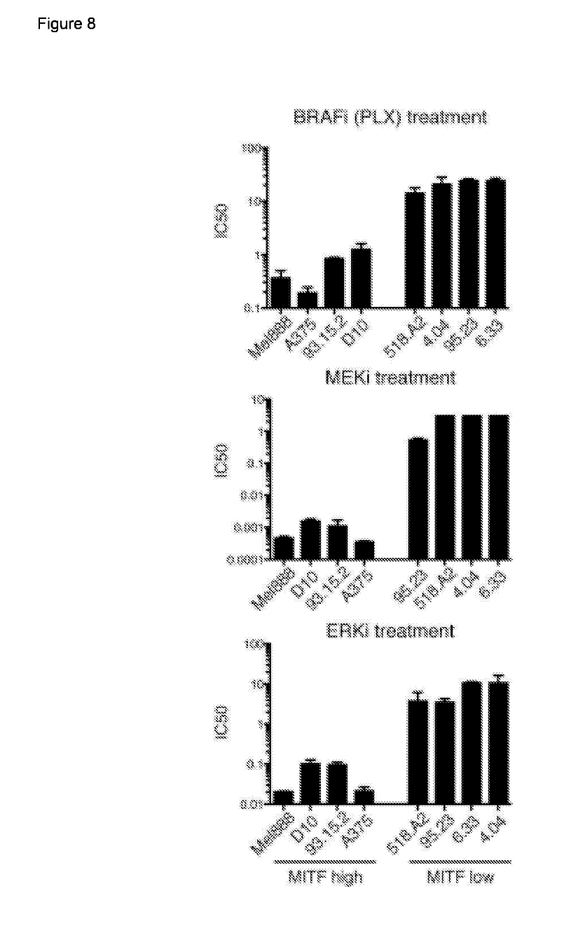

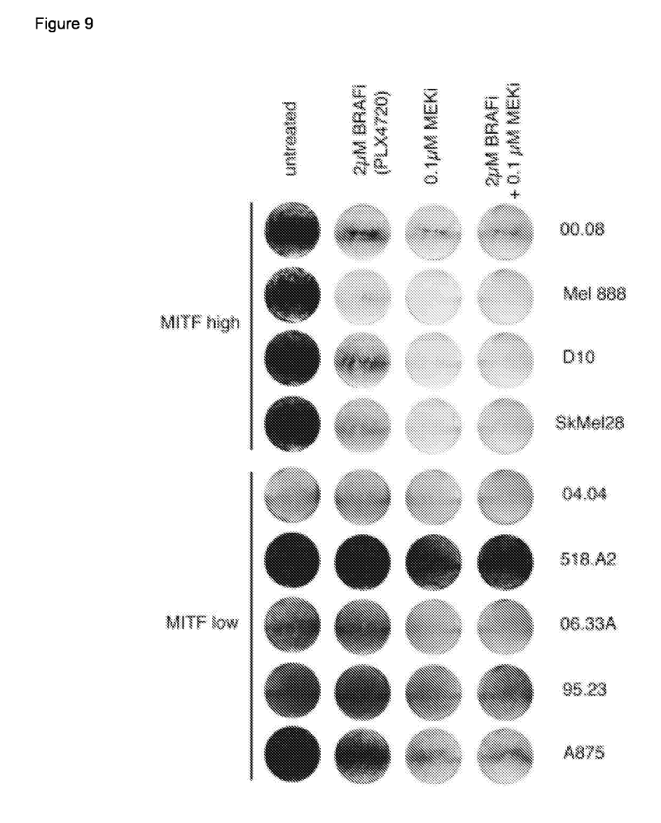

We then determined the spectrum of drug sensitivity among the MITF^{endo}^{_}^{lo} and MITF^{endo}^{_}^{hi} cells. Upon exposure to (a relatively high dose of) 5 μM PLX4720 for six days, there was a clear survival benefit of MITF^{endo}^{_}^{lo} cells, as illustrated by cell proliferation assays and PARP cleavage (FIG. 7). This difference was not due to an insufficient inactivation of the pathway, since ERK phosphorylation was diminished irrespective of MITF status. In agreement, comparing four MITF^{endo}^{_}^{hi} with four MITF^{endo}^{_}^{lo} cell lines, the latter showed a dramatic increase in resistance to MEKi up to 1000-fold, as measured from the IC50 values (FIG. 8). This phenomenon was also seen in combinational inhibition of BRAF and MEK, indicating a much more robust drug resistance phenotype of MITF^{endo}^{_}^{lo} cells than MITF^{endo}^{_}^{hi} cells (FIG. 9).

Seeking whether these findings could be confirmed in an independent dataset, we investigated MITF expression and drug sensitivity in an additional panel of human BRAF^{V600E} mutant melanoma cell lines. Again, we observed an inverse correlation between endogenous MITF expression and the IC50-values for vemurafenib and ERK inhibitor treatment.

Receptor Tyrosine Kinases are Activated and Upregulated in MITF^{endo}^{_}^{lo} Cells

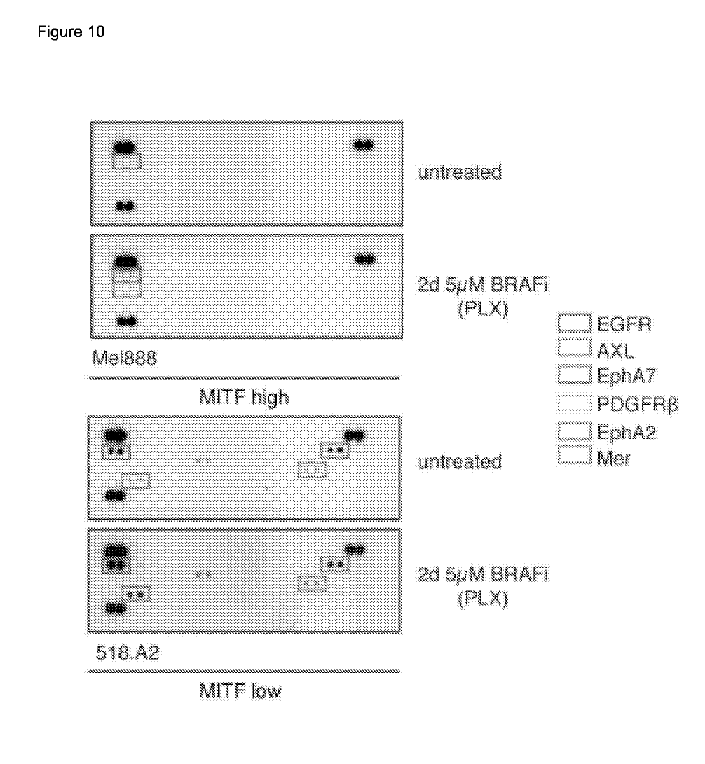

To identify proteins protecting MITF^{endo}^{_}^{lo} cells from MAPK pathway inhibition, we performed RNA sequencing on three MITF^{endo}^{_}^{lo} and three MITF^{endo}^{_}^{hi} BRAF^{V600E} cell lines. We found that, interestingly, several receptor tyrosine kinases (RTKs) were expressed to higher levels in the MITF^{endo}^{_}^{lo} cell lines, including AXL, EGFR and, PDGFRbeta. Encouraged by these findings, we compared the phosphorylation status of RTKs in one MITF^{endo}^{_}^{lo} and one MITF^{endo}^{_}^{lo} cell line. Already in the non-induced state, a subset of RTKs was activated in MITF^{endo}^{_}^{lo} cells. Exposure for two days to 5 μM PLX4720 further increased the activation of several RTKs in MITF^{endo}^{_}^{lo} cells, whereas only a minor activation of RTKs could be detected in MITF^{endo}^{_}^{hi} cells (FIG. 10). In addition to activation, we found increased expression levels of particularly AXL, PDGFR□ and EGFR in MITF^{endo}^{_}^{lo} cells compared to MITF^{endo}^{_}^{hi} cells in a larger set of melanoma cell lines. In an independent dataset we correlated MITF and particular AXL mRNA expression and observed a significant inverse correlation between MITF and AXL expression with a correlation coefficient of −0.84 and a p-value of 6.52E⁻⁰⁹. In addition this dataset confirmed increased resistance to BRAF and ERK inhibition in AXLhigh, MITFlow cells.

These results raise the possibility that the relatively high expression of one or more RTKs contribute to the intrinsically resistant phenotype of MITF^{endo}^{_}^{lo} cells. This prompted us to expose melanoma cells to compounds specifically targeting the RTKs EGFR, AXL and PDGFR□ either alone or in combination. Single inhibition of either AXL or EGFR killed melanoma cells only at relatively high drug concentration irrespective of their MITF expression. In contrast, combined treatment of AXL with MAPK pathway inhibitors did achieve a considerable decrease in cell number (FIG. 11). In a quadruple combination, treatment of MITF^{endo}^{_}^{lo} cells with both AXL, EGFR and PDGFR inhibitors further sensitized MITF^{endo}^{_}^{lo} cells to BRAF or BRAF+MEK inhibition. These results confirm that MITF^{endo}^{_}^{lo} cells are highly intrinsically drug resistant. However, at the same time they unmask several RTKs to contribute to the drug resistance, including AXL, which has not previously been implicated in melanoma drug response.

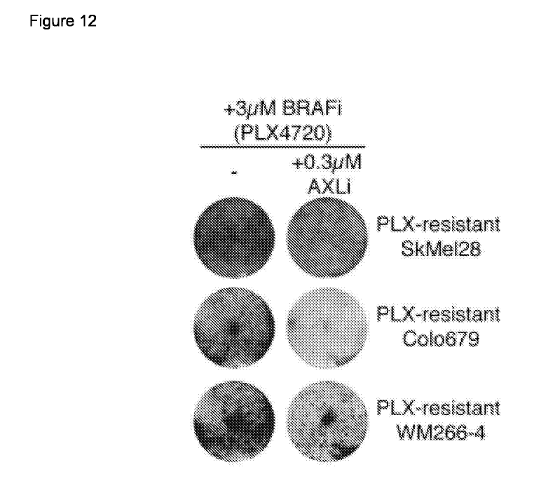

Next, we determined the expression pattern of these RTKs in acquired resistance, particularly in PLX4720-sensitive cells and their resistant counterparts. All three MITF^{acq}^{_}^{loss} cell lines examined (which lost MITF expression after permanent exposure to PLX4720), showed a strong upregulation of AXL, while PDGFR□ and EGFR were upregulated in only one of these cell lines. This was associated with relatively increased phosphorylation of these RTKs. This upregulation of RTKs was not detected in cells that did not lose MITF upon drug resistance. These results suggest that increased expression and activity of several RTKs, particularly AXL, is seen not only in innate but also acquired resistance of melanoma cells. To test if AXL inhibition can be used to effectively target BRAF^{V600E} mutant melanoma cells resistant to MAPK-pathway inhibition, we exposed AXL-expressing PLX4270-resistant cells and their treatment nave counterparts to R428, a previously developed AXL inhibitor. In two out of three resistant cell lines we observe a strong decrease in proliferation/viability upon drug treatment (FIG. 12).

FIG. 13 shows MITF low melanoma cell lines (518.A2, 95.23, A875, 06.33A respectively, per row), also expressing EGFR, exposed to inhibition Braf with either AXii (0.3 μM) (column 2) or EGFRi(2 μM) (column 3) or in a triple combination (Column 4) The first column are untreated cells. After nine days of treatment the remaining cells were stained with crystal violet. The inhibitors were R428 for AXL, Gefitinib for EGFR and PLX4720 for BRAF. Results show that the triple combination surprisingly further inhibit proliferation/viability upon drug treatment, suggesting superior effect compared to the combination of a MAPK inhibitor and AXL inhibitor or to a combination of MAPK inhibitor and an EGFR inhibitor. The cells clearly also rely on expression of EGFR (together with the changes in MITF and AXL).

Taken together this data indicate that receptor tyrosine kinases, including AXL, are new players in resistance of melanoma to MAPK-pathway inhibition and that targeting this RTK either alone or in combination with further RTK inhibitors, in particular AXL inhibitors, together with MAPK-pathway inhibition (e.g. in NRAS, BRAF or KRAS mutated cancers, in particular melanoma), can effectively sensitize innate or acquired resistant melanoma cells to MAPK-pathway inhibition. Indeed first data in other types of mutated cancers, including NRAS mutated cancers like NRAS mutated melanoma confirm this.

1. A combination of an inhibitor of a protein of the MAPK/ERK pathway and an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL for use as a medicament, preferably for use in the treatment of a cancer, preferably melanoma, in a patient.

2. A combination of an inhibitor of a protein of the MAPK/ERK pathway, an inhibitor of a AXL and an inhibitor of a kinase selected from the group consisting of EGFR, PDGFR, IGF-IR, EphA7, PDGFRbeta, EphA2 and Mer, preferably EGFR and PDGFR for use as a medicament, preferably for use in the treatment of a cancer, preferably melanoma, in a patient.

3. The combination of claims 1-2, wherein the cancer in said patient is characterized by the absence of MITF protein, or by a reduced amount of MITF protein.

4. The combination of any one of claims 1-3 wherein the cancer in said patient is characterized by the presence of a kinase selected from the group consisting of AXL, EGFR, PDGFR, IGF-IR, EphA7, PDGFRbeta, EphA2 and Mer, preferably AXL, EGFR and PDGFR, most preferably AXL, or by an increased amount of said kinase.

5. The combination of any one of claims 3-4, wherein expression of the MITF protein and/or the kinase is determined using immune-staining or by determining mRNA levels.

6. The combination of any one of claims 1-5, wherein the cancer is a BRAF-mutated cancer, a NRAS-mutated cancer or a KRAS-mutated cancer, preferably wherein the cancer is a BRAF-mutated melanoma, a NRAS-mutated melanoma or a KRAS-mutated melanoma.

7. The combination of any one of claims 1-6, wherein said inhibitor of a protein of the MAPK/ERK pathway is selected from the group consisting of a RAF-inhibitor, an ERK-inhibitor, and a MEK-inhibitor.

8. The combination of any one of claims 1-7, wherein the inhibitor of the kinase is an inhibitor of AXL.

9. An inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor, for use in treatment of a cancer, preferably melanoma, in a patient, wherein said inhibitor of a protein of the MAPK/ERK pathway is administrated simultaneously, separately or sequentially with an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL or wherein said inhibitor of a protein of the MAPK/ERK pathway is administrated simultaneously, separately or sequentially with an inhibitor of a kinase selected from the group consisting of EGFR, PDGFR, IGF-IR, EphA7, PDGFRbeta, EphA2 and Mer and simultaneously, separately or sequentially with an inhibitor of AXL.

10. An inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL, for use in treatment of a cancer, preferably melanoma, in a patient, wherein said inhibitor of the kinase is administrated simultaneously, separately or sequentially with an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor.

11. The inhibitor for use in treatment of a cancer in a patient according to any one of claims 9-10, wherein the cancer in said patient is characterized by the absence of MITF protein or by a reduced amount of MITF protein.

12. The inhibitor for use in treatment of a cancer in a patient according to any one of claims 9-11, wherein the cancer in said patient is characterized by the presence of a kinase selected from the group consisting of AXL, EGFR, PDGFR, IGF-IR, EphA7, PDGFRbeta, EphA2 and Mer, preferably AXL, EGFR and PDGFR, most preferably AXL, or by an increased amount of said kinase, or wherein the cancer in said patient is characterized by the presence of AXL and a kinase selected from the group consisting of EGFR, PDGFR, IGF-IR, EphA7, PDGFRbeta, EphA2 and Mer, preferably, EGFR and PDGFR, or by an increased amount of said kinase and AXL.

13. The inhibitor for use in treatment of a cancer in a patient according to any one of claims 9-12, wherein MITF protein and/or the kinase is determined using immune-staining or by determining mRNA levels.

14. The inhibitor for use in treatment of a cancer in a patient according to any one of claims 9-13, wherein the cancer is a BRAF-mutated cancer, a NRAS-mutated cancer or a KRAS-mutated cancer, preferably wherein the cancer is a BRAF-mutated melanoma, a NRAS-mutated melanoma or a KRAS-mutated melanoma.

15. A product comprising an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor, and an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL, as a combined preparation for simultaneous, separate or sequential use in treatment of a cancer, preferably melanoma, in a patient, or comprising an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor, and an inhibitor of AXL and an inhibitor of a kinase selected from the group consisting of EGFR, PDGFR, IGF-IR, EphA7, PDGFRbeta, EphA2 and Mer, preferably EGFR and PDGFR, as a combined preparation for simultaneous, separate or sequential use in treatment of a cancer, preferably melanoma, in a patient.

16. A method for the treatment of a cancer, preferably melanoma, in a patient, wherein the method comprises the simultaneous, separate or sequential administering to the patient of an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor, and an inhibitor of a kinase selected from the group consisting of AXL, EGFR, PDGFR, IGF-IR, EphA7, PDGFRbeta, EphA2 and Mer, preferably AXL, EGFR and PDGFR, most preferably AXL or wherein the method comprises the simultaneous, separate or sequential administering to the patient of an inhibitor of a protein of the MAPK/ERK pathway, preferably wherein said inhibitor of a protein of the MAPK/ERK pathway is selected from the group consisting of a RAF-inhibitor, an ERK-inhibitor, and a MEK-inhibitor, and an inhibitor of AXL and an inhibitor of a kinase selected from the group consisting of EGFR, PDGFR, IGF-IR, EphA7, PDGFRbeta, EphA2 and Mer, preferably EGFR and PDGFR.

17. The method according to claim 16, wherein the cancer in said patient is characterized by the absence of MITF protein or by a reduced amount of MITF protein.

18. The method according to any one of claims 16 - 17 wherein the cancer in said patient is characterized by the presence of a kinase selected from the group consisting of AXL, EGFR, PDGFR, IGF-IR, EphA7, PDGFRbeta, EphA2 and Mer, preferably AXL, EGFR and PDGFR, most preferably AXL, or by an increased amount of said kinase, or wherein the cancer in said patient is characterized by the presence of AXL and a kinase selected from the group consisting of EGFR, PDGFR, IGF-IR, EphA7, PDGFRbeta, EphA2 and Mer, preferably, EGFR and PDGFR, or by an increased amount of said kinase and AXL.

19. The method according to any one of claims 16-18, wherein MITF protein and/or the kinase is determined using immune-staining or by determining mRNA levels.

20. The method according to any one of claims 16-19, wherein the cancer is a BRAF-mutated cancer, a NRAS-mutated cancer or a KRAS-mutated cancer, preferably wherein the cancer is a BRAF-mutated melanoma, a NRAS-mutated melanoma or a KRAS-mutated melanoma.

21. A method for the diagnosis of cancer of a patient, the method comprising the steps of
(a) determining the level of MITF in the cancer cell(s) obtained form said patient; and
(b) determining the level of a kinase and/or the phosphorylation status selected from the group consisting of AXL, EGFR, PDGFR, IGF-IR, EphA7, PDGFRbeta, EphA2 and Mer, preferably AXL, EGFR and PDGFR, most preferably AXL, in the cancer cell(s) obtained from said patient, preferably wherein at least AXL and at least one other kinase is determined.

22. The method of claim 21, comprising the step of determining whether the cancer cell(s) obtained from said patient are BRAF-mutated, a NRAS-mutated cancer or KRAS-mutated cancer cells.

23. A method for predicting treatment response of a cancer, preferably melanoma, of a patient, the method comprising the step of determining the amount of MITF in cancer cell(s) obtained form said patient and;
and determining a predicted treatment response based on the determined amount of MITF, wherein the absence of MITF protein or a reduced amount of MITF indicates a bad predicted treatment response.

24. The method of claim 23, wherein the method in performed in vitro.
